(19) United States
(12) Patent Application Publication
(10) Pub. No.: US 2015/0087619 A1
(43) Pub. Date: Mar. 26, 2015

(54) METHOD OF MITIGATING VIRUS ASSOCIATED END-ORGAN DAMAGE

(71) Applicant: CHIMERIX, INC., Durham, NC (US)

(72) Inventors: George R. Painter, JR., Atlanta, GA (US); Ernest Randall Lanier, Chapel Hill, NC (US); Dorothy Margolskee, Voorhees, NJ (US); Gwendolyn Powell Painter, Atlanta, GA (US); Roy Ware, Raleigh, NC (US)

(21) Appl. No.: 14/397,471

(22) PCT Filed: Apr. 26, 2013
(86) PCT No.: PCT/US13/38351

§ 371 Date: Oct. 27, 2014

Related U.S. Application Data
(60) Provisional application No. 61/639,764, filed on Apr. 27, 2012.
(60) Provisional application No. 61/672,666, filed on Jul. 17, 2012.
(60) Provisional application No. 61/677,286, filed on Jul. 30, 2012.
(60) Provisional application No. 61/684,524, filed on Aug. 17, 2012.
(60) Provisional application No. 61/696,524, filed on Sep. 4, 2012.

(51) Int. Cl. A61K 31/675

(52) U.S. Cl. CPC A61K 31/675
USPC 514/86

(57) ABSTRACT

The present application provides methods and compositions for treatment or reducing risk of dsDNA virus infection in post-hematopoietic cell transplant (HCT or HSCT) patients. This application also provides methods and composition for reducing the incidence of BK virus associated hematuria and/or renal impairment. The invention also relates to a method of lowering BK viral load in post-hematopoietic cell transplant (HCT or HSCT) patients with HDP-CDV (HDP-CDV) or compound of Formula II, or a pharmaceutically acceptable salt thereof, thereby delaying onset of or reducing risk of end organ disease in these patients.

### RELATED APPLICATIONS

This application claims priority to and the benefit of U.S. Provisional Application Nos. 61/639,764, filed Apr. 27, 2012; 61/672,666, filed Jul. 17, 2012; 61/677,286, filed Jul. 30, 2012; 61/684,524, filed Aug. 17, 2012; and 61/696,524, filed Sep. 4, 2012, each of which is incorporated by reference herein in its entirety.

### FIELD OF THE INVENTION

The present invention concerns methods and composition for reducing the incidence of BK virus associated end-organ damage, e.g., hematuria and/or renal impairment.

### BACKGROUND

BK Virus (“BKV”) is a polyomavirus; a small, non-enveloped, dsDNA virus, which was first isolated in 1971 from a renal transplant patient (initials BK) with ureteric stenosis. Primary BKV infection normally occurs early in life presenting as a mild disease with flu-like symptoms. Following primary infection, the virus establishes lifelong latency in urogenital epithelial cells, rarely causing disease in healthy adults. Conversely, BKV causes significant disease in patients with prolonged immunosuppression, such as BKV associated nephropathy (BKVAN) in renal transplant patients and hemorrhagic cystitis (“HC”) in hematopoietic [stem] cell transplant (“HCT” or “HSCT”) recipients.

BKV infection is associated with HC in approximately 30% (5-60%) of HSCT recipients (the risk of HC is increased after myeloablative conditioning or mismatched donor HCT). Although rarely fatal, HC episodes can be severe; very painful; associated with significant hematuria and clotting; may prolong hospitalization; and can result in end-organ damage, e.g., impairment of kidney and/or bladder function.

There is a need for improved methods and compositions to treat BKV infections and prevention of BKV associated end-organ damage in HSCT patients. The present application provides methods and compositions for improving the bioavailability of anti-viral agents for treatment of BKV infection associated HC and hematuria (Hem+), renal impairment and BK neuropathy.

### SUMMARY OF THE INVENTION

The present application relates to methods and compositions for the delaying onset of, reducing risk of, or treatment of end-organ damage or impairment in a patient infected with BK virus (BKV), by orally administering to the subject a therapeutically effective dose of a compound of Formula:

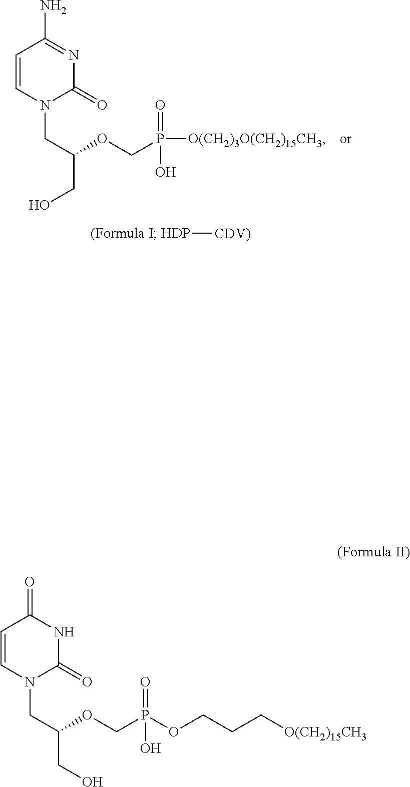

or a pharmaceutically acceptable salt thereof.

In some embodiments of the current invention, the subjects treated may be stem cell transplant or renal transplant recipients. In an embodiment, the subject may be a post-hematopoietic stem cell transplant (HSCT) subject.

The end-organ damage or impairment for treatment, delaying onset, reducing risk, according to embodiments of the current invention, may include, e.g., without being limited to: kidney, ureter, urinary bladder, prostate, and/or urethra. Microscopic hematuria, may reflect kidney damage or disorder, as defined with heme+urinalyses; renal impairment may be defined as having an elevated creatinine (≧120 μmol/L) on the last measurement during treatment that was also ≧25% increased from baseline.

The present application also relates to methods and compositions for reducing the incidence of BK virus associated hematuria and/or renal impairment. The pharmaceutical composition of the current invention may prevent the emergence of hematuria and renal impairment, both associated with end-organ damage from BK virus infection. The invention also relates to a method of prevention of BK viral load increase in post-HSCT patients with HDP-CDV, thereby preventing end organ disease in these patients. The pharmaceutical agent of the current invention may prevent end-organ damage or impairment, for example, kidney, ureter, urinary bladder, prostate, and urethra damage or impairment.

The current invention provides a method of delaying onset of, reducing risk of a disease or disorder in a subject at risk of virus infection reactivation, by orally administering to the subject a pharmaceutical composition of a therapeutically effective dose of a compound of Formula I or II:

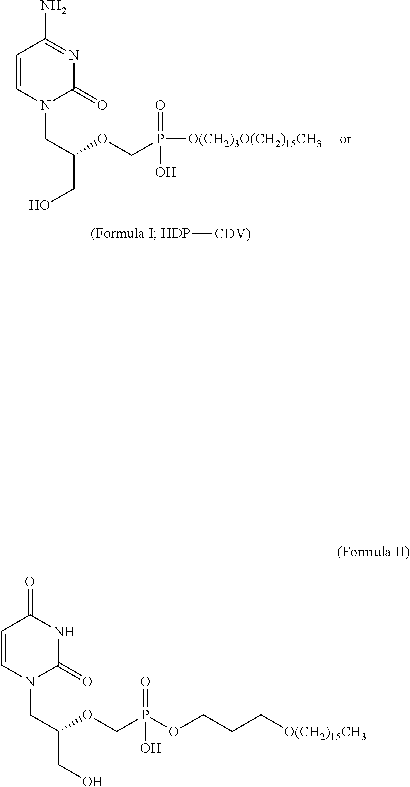

or a pharmaceutically acceptable salt thereof, wherein the virus at risk of reactivation may be BK virus.

In some embodiments, the subject at risk of virus infection reactivation may be a stem cell transplant or renal transplant recipients. In an embodiment, the subject may be a post-hematopoietic stem cell transplant (HSCT) subject.

In yet another embodiment, the method of the current invention reduces the risk and/or delays onset of hematuria or renal impairment in a post-HSCT subject or subjects after renal transplantation. The prevention of hematuria or renal impairment in post-HSCT patient may be associated with prevention of viral reactivation in the subject. In one embodiment, the prevention of virus infection reactivation prevents hematuria or renal impairment in said subject.

The embodiments of the current invention also provide that the administration of the pharmaceutical composition or pharmaceutically acceptable salt of HDP-CDV prevents viral load increase in a patient at risk of virus infection reactivation. The virus, according to this embodiment, may be BK virus.

In an embodiment of the current invention, the delaying onset or reducing risk of viral load increase in a patient at risk of virus infection reactivation prevents hematuria and/or renal impairment.

The invention, according to the current embodiments, provides that the pharmaceutical composition of the current invention delays onset or reduces risk of Hematuria (Hem+) in HSCT patients with BKV at base line (BKU+), compared to the placebo group. The invention also provides that no significant difference in Hem+ may be observed in patients who are BKV viruria negative (BKU−) at baseline compared to the placebo group. See FIG. 1.

The current invention provides that the pharmaceutical composition of the current invention delays onset or reduces risk of increase in creatinine level and worsening of renal function in patients who may be BKV viruria (BKU+) at baseline (post-HSCT engraft) compared to the placebo group. The invention further provides that the pharmaceutical composition of the current invention may not impact the end organ damage, for example kidney, ureter, urinary bladder, prostate, and urethra damage, in patients who may be BKV viruria negative (BKU−) at baseline. In these patients, the creatinine level may not increase compared to the placebo group. See FIG. 2.

The invention also provides that when BKU+ patients are compared for their maximum BKV viruria, patients receiving the pharmaceutical composition of the current invention may have both a reduction in the BKV viral load and a reduced risk or incidence of end organ damage, for example kidney, ureter, urinary bladder, prostate, and urethra damage, when compared to the placebo group. The invention also provides that among patients who develop end organ disease after treatment, the viral load may be significantly higher compared to patients who may not develop end organ disease. See FIG. 3. Moreover, patients who developed end organ disease, for example kidney, ureter, urinary bladder, prostate, and urethra damage, after treatment with HDP-CDV may have significantly lower viral load compared to patients who received placebo. See FIG. 3.

In some embodiments, the subject may be treated with about 100 mg of the compound(s) of the current invention, BIW. In additional embodiments, the subject may be treated once a week (QW) or twice a week (BIW) with about 150 mg or about 200 mg. A subject infected with a dsDNA virus, e.g., BKV, may be treated once a week (QW) with about 200 mg or twice a week (BIW) with about 100 mg of a compound of Formula I or Formula II. In further embodiments, the subject may be treated with once a week (QW) with about 150 mg or about 200 mg, or twice a week (BIW) with about 75 mg or about 100 mg of a compound of Formula I or Formula II.

In one embodiment, a method of treating, reducing risk of, or time-to-onset of a viral infection and/or viral infection associated disease or disorder, e.g., BKV infection, is provided, the method comprising administering an effective amount of a compound of Formula I or Formula II, or salt, ester or derivative thereof, optionally in combination with one or more bioavailability enhancing compounds to a host in need thereof. The compositions may be administered in an effective amount for the treatment or prophylaxis of a host infected with a virus, such as BKV, optionally in combination with a pharmaceutically acceptable carrier. The compounds or compositions may be administered, e.g., orally or parenterally.

In one embodiment, a method of treating, reducing risk of, or delaying time-to-onset of a viral infection and/or viral infection associated disease or disorder (e.g., a polyomavirus BK infection, JC virus infection, Epstein-Barr virus infection, herpes simplex virus infection, human herpes virus 6 infection, vaccinia virus infection, molluscum contagiosum virus infection) is provided, the method comprising administering an effective amount of a compound of Formula I or Formula II, or salt, ester or derivative thereof, optionally in combination with one or more bioavailability enhancing compounds to a host in need thereof. The compositions may be administered in an effective amount for the treatment or prophylaxis of a host infected with a virus, such as a polyomavirus BK, optionally in combination with a pharmaceutically acceptable carrier. The compounds or compositions are administered, e.g., orally or parenterally.

A method of delaying onset or reducing risk of spread of Varicella-Zoster Virus (“VZV”) in a subject is provided, where the method comprises orally administering to the subject a pharmaceutical composition comprising a therapeutically effective dose of a compound selected from:

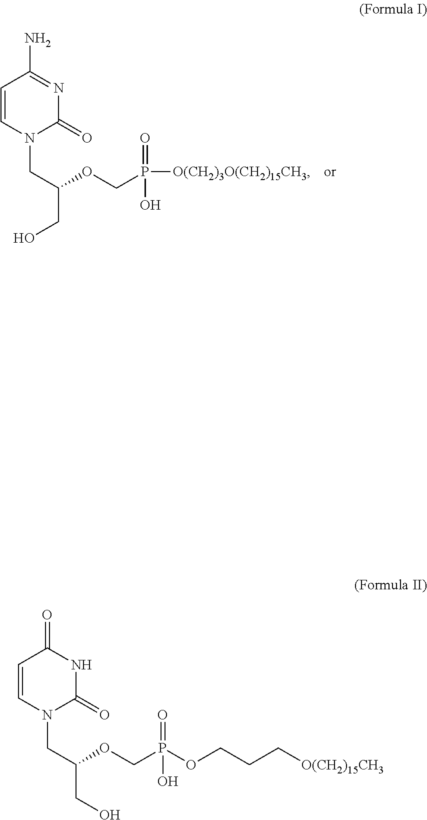

or a pharmaceutically acceptable salt thereof.

The method of preventing spread of VZV may involve administered 1.25 mg/kg, 2.5 mg/kg, 5.0 mg/kg, 10 mg/kg, or 20 mg/kg of the compound of Formula I or Formula II, on day 1, 2, or 4 after post-hematopoietic stem cell transplant (HSCT).

In one particular embodiment, a composition is provided that includes a cidofovir lipid derivative drug, administered to a subject in an effective amount for the treatment of a viral infection, such as a BK virus infection. In one embodiment, the nucleoside derivative is an alkoxyalkyl ester of cidofovir, such as an alkoxyalkanol of cidofovir (HDP-cidofovir or HDP-CDV). For example, the compound has the structure:

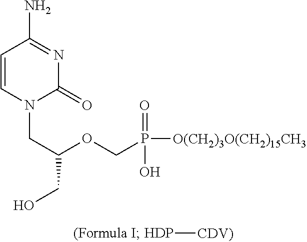

HDP-CDV, a broad spectrum lipid acyclic nucleoside phosphonate converted intracellularly into the active antiviral cidofovir diphosphate (CDV-PP), may have a long intracellular half-life of ˜6.5 days.

In one embodiment, the nucleoside derivative of the current invention is obtained by deamination or hydrolysis of an alkoxyalkyl ester of cidofovir, such as an alkoxyalkanol of cidofovir (HDP-cidofovir or HDP-CDV). For example, the compound derived by deamination or hydrolysis of HDP-CDV has the following structure:

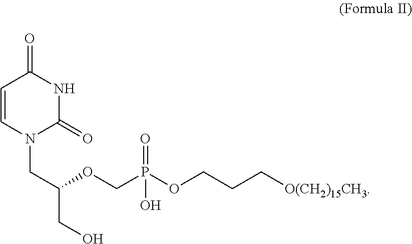

### BRIEF DESCRIPTION OF THE DRAWINGS

FIG. 1 illustrates line graphs of data showing that HDP-CDV reduces hematuria in subjects with BKV.

FIG. 2 illustrates line graphs of data showing that HDP-cidofovir reduces renal impairment in subjects with BKV.

FIG. 3 illustrates the relationship between maximum BKV viruria and end organ damage.

### DETAILED DESCRIPTION OF THE INVENTION

The pharmaceutical composition of the current invention reduces risk of and/or delays onset of end-organ damage, e.g., the emergence of hematuria and renal impairment, which are associated with end organ (for example kidney, ureter, urinary bladder, prostate, and urethra) damages from BK virus infection. The invention also relates to a method of reducing risk of and/or delaying onset of BK viral load increase in post-HSCT patients with the compound of Formula I or Formula II, thereby reducing the risk of and/or delaying the onset of end organ disease in these patients. In one embodiment, the invention provides preventing Varicella-Zoster Virus (“VZV”) spread with the compound of Formula I or Formula II.

### Method of Prevention or Treatment of End-Organ Damage

The present application relates to methods for the reducing risk of and/or delaying onset of, or treatment of end-organ damage or impairment in a patient infected with BK virus, by orally administering to the subject a therapeutically effective dose of a compound of Formula:

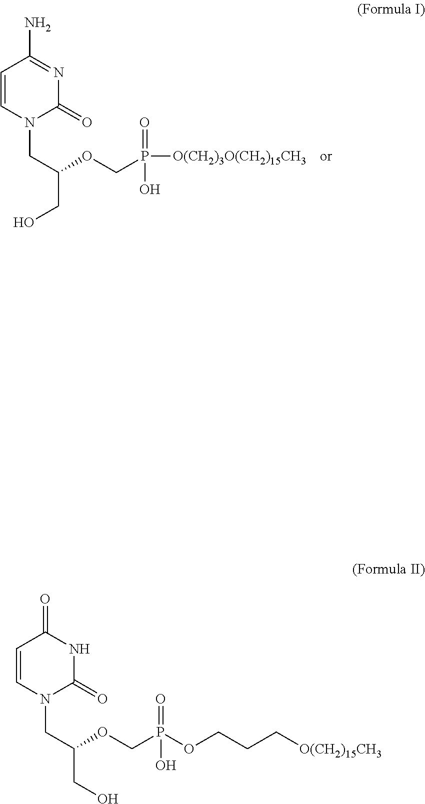

or a pharmaceutically acceptable salt thereof.

In some embodiments of the current invention, the method may provide that subjects for treatment or reducing risk of and/or delaying onset of end-organ damage or impairment may be stem cell transplant or renal transplant recipients. In one embodiment, the subject may be a post-hematopoietic stem cell transplant (HSCT) subject. In yet other embodiments, the subject may be islet cell transplant recipient, bone marrow transplant recipient, endothelial cell transplant recipient, epidermal cell transplant recipient, myoblast transplant recipient, muscle derived stem cell recipient, and/or neural stem cell transplant recipient.

The end-organ damage or impairment for treatment or prevention, according to embodiments of the current invention may include kidney, ureter, urinary bladder, prostate, and/or urethra.

In some embodiments, the method provides that about 100 mg of the compound(s) of the current invention may be administered once a week (QW) or twice a week (BIW) to a subject for prevention or treatment of end-organ damage or impairment. The subject may be treated QW or BIW with about 150 mg or about 200 mg. A subject infected with a dsDNA virus, e.g., BKV, may be treated once a week (QW) with about 200 mg or twice a week (BIW) with about 100 mg of a compound of Formula I or Formula II. In further embodiments, the subject may be treated with once a week (QW) with about 150 mg or about 200 mg, or twice a week (BIW) with about 75 mg or about 100 mg of a compound of Formula I or Formula II.

In yet other embodiments, a subject may be treated with about 50-99 mg, 101-149 mg, 151-199 mg, 201-250 mg, or >251 mg dose without resulting in significant adverse effects (AEs). In some embodiments, the dose in mg may vary within one week, two weeks, or during the entire treatment period.

The relationship between maximum BKV viruria and end organ damage by treatment group may be determined. HDP-CDV or the compound of Formula II may be effective in reducing BKV viral load and the risk or incidence of end organ damage in BKU+ patients. Among patients who developed end organ disease after treatment (33%), the viral load may be significantly higher compared to patients (e.g., 67%) who did not develop end organ disease (e.g., 3.2×10⁸ vs. 2.0×10⁸). Moreover, patients who developed end organ disease after treatment with HDP-CDV may have significantly lower viral load compared to patients who received placebo.

Method for Reducing the Incidence of BKV Associate Hematuria and/or Renal Impairment

The present application also relates to methods for reducing the incidence of BK virus associated hematuria and/or renal impairment. The methods of the current invention may prevent the emergence of hematuria and renal impairment, both associated with end-organ damages from BK virus infection. The invention also relates to a method of reducing risk of and/or delaying onset of BK viral load increase in post-HSCT patients with HDP-CDV, thereby reducing risk of and/or delaying onset of end organ disease in these patients. The pharmaceutical composition of the current invention may prevent end-organ damage or impairment, for example, kidney, ureter, urinary bladder, prostate, and urethra damage or impairment.

In some embodiments, the methods for reducing the incidence of BK virus associated hematuria and/or renal impairment may provide that about 100 mg of the compound(s) of the current invention may be administered once a week (QW) or twice a week (BIW) to a subject for prevention or treatment of end-organ damage or impairment. The subject may be treated QW or BIW with about 150 mg or about 200 mg. A subject infected with a dsDNA virus, e.g., BKV, may be treated once a week (QW) with about 200 mg or twice a week (BIW) with about 100 mg of a compound of Formula I or Formula II. In further embodiments, the subject may be treated with once a week (QW) with about 150 mg or about 200 mg, or twice a week (BIW) with about 75 mg or about 100 mg of a compound of Formula I or Formula II.

In yet other embodiments, the methods for reducing the incidence of BK virus associated hematuria and/or renal impairment may provide that a subject may be treated with about 50-99 mg, 101-149 mg, 151-199 mg, 201-250 mg, or >251 mg dose without resulting in significant adverse effects (AEs). In some embodiments, the dose in mg may vary within one week, two weeks, or during the entire treatment period.

The impact of HDP-CDV or the compound of Formula II on hemorragic cystitis emergence may be assessed based on the incidence of treatment emergent hematuria. HDP-CDV may prevent Hematuria (Hem+) in HSCT patients who are BKV positive at base line (BKU+). There may not be a significant difference in Hem+ in patients who are BKV viruria negative (BKU−) at baseline compared to the placebo group.

The impact of HDP-CDV or the compound of Formula II on renal dysfunction in subjects with preexisting BKV infection may be measured. HDP-CDV or the compound of Formula II may prevent increase in creatinine level and worsening of renal function in patients who were BKV viruria (BKU+) at baseline (post-HSCT engraft) compared to the placebo group. HDP-CDV may not impact the end organ damage in patients who were BKV viruria negative (BKU−) at baseline. In these patients, the creatinine level may not increase compared to the placebo group.

The methods of the current invention provides that compound of Formula I or Formula II, or a pharmaceutically acceptable salt thereof, may be associated with reduction of microscopic hematuria in subjects shedding BKV in their urine. Subjects who had BKV viruria during treatment period, i.e., receiving compound of Formula I or Formula II, or a pharmaceutically acceptable salt thereof, may have a 2-10 fold decrease in blood positive urinalysis compared to subjects receiving placebo. In some embodiments, between the treatment and placebo groups, the difference in blood positive urinalysis may be 2-8, 2-7, 2-6, 2-5, or 2-4 fold. Among subjects without BK viruria, the rates of blood positive urinalysis may be low or comparable between the treated versus the untreated, e.g., patients receiving placebo.

The methods of treatment with HDP-CDV or compound of Formula II, or a pharmaceutically acceptable salt thereof, may have beneficial effect on BK associated bladder events. For example, high BK viruria measurements (e.g., ≧1×10¹⁰ copies/mL) may be associated with clinically important events (e.g., AEs for cystitis or blood in urine). Compared to placebo-treated subjects, the rates of confirmed blood positive urinalyses may occur at 1/10^{th}, 1/9^{th}, ⅛^{th}, 1/7^{th}, ⅙^{th}, ⅕^{th}, ¼^{th} the rate in HDP-CDV or compound of Formula II treated subjects. In some embodiments, the incidence of sustained BK viruria may be reduced for HDP-CDV or Formula II, or a pharmaceutically acceptable salt thereof, treated subjects who developed BK viruria during treatment.

The methods of the current embodiments may involve measuring serum creatinine concentrations as a marker of renal function. The current methods may measure kidney function by calculating creatinine clearance from the body by the kidneys. This is referred to as creatinine clearance and it estimates the rate of filtration by kidneys (glomerular filtration rate, or GFR). The creatinine clearance can be measured in two ways. It can be calculated by a formula using serum (blood) creatinine level, patient's weight, and age. Creatinine clearance can also be more directly measured by collecting a 24-hour urine sample. Normal level of creatinine in blood is 0.7 to 1.3 mg/dL for men and 0.6 to 1.1 mg/dL for women. See Creatinine—Blood, Medline Plus, U.S. National Library of Medicine, NIH. If kidney function is abnormal, creatinine levels will increase in the blood (because less creatinine is released through your urine).

Creatinine level more than about 1.36 mg/mL in urine may be considered elevated. In the methods of the current invention, about 15% or about 25% increase in creatinine level from baseline is considered clinically important change during the treatment period.

The current method provides evaluation of microscopic hematuria using heme +1 urinalysis as a surrogate. End of treatment (last value) elevations in serum creatinine measurements (e.g., >120 microM (1.36 mg/dl)) may be considered clinically meaningful. Pre-existing renal dysfunction may be distinguished by measuring both the last value for creatinine, which is higher than the normal level, e.g., >120 microM and at least 15% or 25% increase from baseline.

The methods of the current invention provide reducing the risk of or delaying onset of end-organ damage in BKV positive patients by oral administration of HDP-CDV or compound of Formula II, or pharmaceutically acceptable salt thereof. Subjects, who are BK viruric during the treatment period, may show beneficial effect due to treatment with HDP-CDV or compound of formula II, in reducing the incidence of renal dysfunction (creatinine elevations) by 1.5-4.5 fold. The incidence of renal dysfunction may be reduced by about 1.6, 1.7, 1.8, 1.9, 2.0, 2.1, 2.2, 2.3, 2.4, 2.5, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 fold. Among the BK-positive subjects of the current invention, there may be a 1.2-4.4 fold decrease of creatinine elevations or new onset heme+urinalyses. Among subjects who remain BK negative during the treatment period, the rates for either creatinine elevations or the combined analysis of creatinine or heme+urine may be numerically similar.

BK virus can have effects on renal function and the bladder (hematuria, cystitis, dysuria etc.). An analysis of routine laboratory values (serum creatinine elevations and the presence of new onset, confirmed hematuria) are potential markers of BK effects in these subjects post HSCT. In the clinical study of the current invention, baseline data and initial creatinine levels of subjects who are BK viruria positive at some point during treatment and subjects who are consistently BK negative may be measured. BKV-positive subjects may have higher baseline level of creatinine compared to the BKV-negative subjects. While there may be similar frequencies of creatinine elevations between BK-positive and BK-negative subjects, the distributions within the BK positive group with respect to HDP-CDV- and placebo-treatment may be different.

In one embodiment about 1.2-5.0 fold difference in frequency of hematuria between BK positive subjects compared to untreated subjects was observed. The method of the current invention provides measuring last value of creatinine, % increase over baseline level of creatinine, and heme +1 urinalysis during the treatment period among subjects treated with compound of Formula I or Formula II or pharmaceutically acceptable salt thereof.

### Methods for Preventing Disease or Disorder Due to Virus Reactivation

The current invention also provides a method of preventing a disease or disorder in a subject at risk of virus infection reactivation, by orally administering to the subject a pharmaceutical composition of a therapeutically effective dose of a compound of Formula I or Formula II:

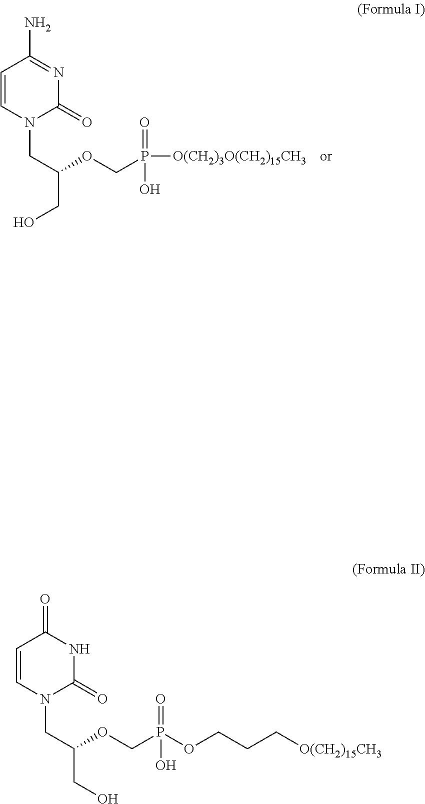

or a pharmaceutically acceptable salt thereof, wherein the virus at risk of reactivation may be BK virus.

In one embodiment, the subject at risk of virus infection reactivation may be stem cell transplant or renal transplant recipients. In an embodiment, the subject may be a post-hematopoietic stem cell transplant (HSCT) subject. In yet other embodiments, the subject may be islet cell transplant recipient, bone marrow transplant recipient, endothelial cell transplant recipient, epidermal cell transplant recipient, myoblast transplant recipient, muscle derived stem cell recipient, and/or neural stem cell transplant recipient.

In yet other embodiments, the subject may be islet cell transplant recipient, bone marrow transplant recipient, endothelial cell transplant recipient, epidermal cell transplant recipient, myoblast transplant recipient and/or neural stem cell transplant recipient.

In yet another embodiment, the method of the current invention prevents hematuria or renal impairment in a post-HSCT subject. The prevention of hematuria or renal impairment in post-HSCT patient may be associated with prevention of viral reactivation in the subject. In one embodiment, the prevention of virus infection reactivation prevents hematuria or renal impairment in said subject.

### Compositions for Prevention or Treatment of End-Organ Damage

In one particular embodiment, a composition may be provided that includes a cidofovir lipid derivative, administered to a subject in an effective amount for the treatment of a viral infection, such as BK virus infection. In one embodiment, the nucleoside derivative may be an alkoxyalkyl ester of cidofovir, such as an alkoxyalkanol of cidofovir (HDP-cidofovir or HDP-CDV). For example, the compound has the structure:

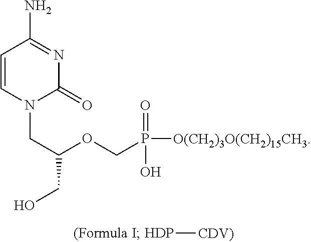

In one embodiment, a composition may be provided that includes a nucleoside derivative of the current invention, obtained by deamination or hydrolysis of HDP-CDV alkoxyalkyl ester of cidofovir, such as an alkoxyalkanol of cidofovir (HDP-cidofovir or HDP-CDV). For example, the compound derived by deamination or hydrolysis of HDP-CDV has the structure:

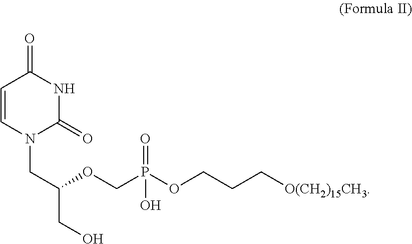

The embodiments of the current invention also provide that the administration of the pharmaceutical composition or pharmaceutically acceptable salt of HDP-CDV or the compound of Formula II may prevent viral load increase in a patient at risk of virus infection reactivation. The virus, according to this embodiment, may be BK virus. In an embodiment of the current invention, the prevention of viral load increase in a patient at risk of virus infection reactivation may prevent hematuria or renal impairment.

The pharmaceutical composition or pharmaceutically acceptable salt of HDP-CDV or the compound of Formula II of the invention may reduce the incidence of BKV events, despite a high incidence of BK viruria in subjects at the time of enrollment, and persisting during the active treatment period. Treatment with Formula I or II may reduce reported BKV-related bladder AEs (adverse effects/events), hemorrhagic cystitis, or hematuria. Treatment with Formula I or II may decrease in overall incidence of blood positive urinalysis; and may reduce in the incidence of sustained, new onset BK viruria during the treatment period.

In some embodiments, certain doses of the compound(s) (about 200 mg QW, about 100 mg BIW, and about 200 mg BIW) may be superior to placebo in reducing the proportion of subjects reaching a composite endpoint of initiation of a second anti-viral therapy or viral disease or viral dsDNAemia of >1000 copies/mL. In one embodiment, the invention provides delaying onset of, reducing risk of onset, or treating viral disease and/or infection in subjects who received about 200 QW, 100 mg BIW, or 100 mg QW of the compound(s). The virus infection and/or virus associated disease or disorder may be BK virus infection or BK virus related disease or disorder.

In an embodiment of the current invention, except for a dose of about 40 mg QW, other HDP-CDV doses and dose regimens of the current invention may have better antiviral activity when compared to placebo. Activity of the compound may increase with dose and/or dose frequency.

The embodiments of the current invention provide delaying onset of, reducing risk of onset, viral infected associated end-organ disease or impairment in subjects who received about 200 QW, 100 mg BIW, or 100 mg QW of the compound(s). In an embodiment about 40 mg QW dose may be inactive. The embodiments of the current invention provide a dose-dependent treatment protocol.

The invention, according to the current embodiments, provides that the pharmaceutical composition of the current invention may prevent Hematuria (Hem+) in HSCT patients with BKV at base line (BKU+), compared to the placebo group. The invention also provides that no significant difference in Hem+ may be observed in patients who are BKV viruria negative (BKU−) at baseline compared to the placebo group.

The current invention provides that the pharmaceutical composition of the current invention prevents increase in creatinine level and worsening of renal function in patients who may be BKV viruria (BKU+) at baseline (post-HSCT engraft) compared to the placebo group. The invention further provides that the pharmaceutical composition of the current invention may not impact the end organ damage, for example kidney, ureter, urinary bladder, prostate, and urethra damage, in patients who may be BKV viruria negative (BKU−) at baseline. In these patients, the creatinine level may not increase compared to the placebo group.

The invention also provides that when BKU+ patients are compared for their maximum BKV viruria, patients receiving the pharmaceutical composition of the current invention may have both a reduction in the BKV viral load and a reduced risk or incidence of end organ damage, for example kidney, ureter, urinary bladder, prostate, and urethra damage, when compared to the placebo group. The invention also provides that among patients who develop end organ disease after treatment, the viral load may be significantly higher compared to patients who do not develop end organ disease. Moreover, patients who developed end organ disease, for example kidney, ureter, urinary bladder, prostate, and urethra damage, after treatment with HDP-CDV may have significantly lower viral load compared to patients who received placebo. According to embodiments of the current invention, patients who may develop end organ disease after treatment, the viral load may be significantly higher compared to patients who did not develop end organ disease. For example, without being limited to the specific numbers described herein, viral load may be, e.g., 3.2×10⁸ in end-organ damaged or impaired patients vs., e.g., 2.0×10⁸ in subjects who do not develop the disease.

The embodiments of the current invention provide that there may be no indication of nephrotoxicity or myelotoxicity associated with HDP-CDV administration, regardless of dose and dosing frequency. HDP-CDV dose regimens of about 40 mg QW and about 100 mg QW may have tolerability profiles similar to placebo in terms of AEs and laboratory abnormalities. A dose-related increase in ALT may be associated with HDP-CDV therapy.

In additional embodiments, the current invention provides the time to onset of viral DNAemia (e.g., BK DNAemia) may be delayed in subjects, who may be negative for the viral DNA at baseline and received about 100 mg QW, about 200 mg BIW, and about 100 mg BIW of the compound(s) of the current invention, as compared to placebo. The time to onset may be delayed by several days, e.g., 2-10 days, 3-11 days, 4-12 days, 5-13 days, 6-14 days, 7-15 days, 8-16 days, 9-17 days, 10-18 days, 11-19 days, 12-20 days, 13-21 days, 14-22 days, 15-23 days, 16-24 days, 17-25 days, or >25 days.

In subjects treated by the methods of the invention, the virus does not develop mutations, e.g., UL97 and/or UL54 mutations, for drug resistance.

In specific embodiments, the current invention provides that no mutation in UL54 and UL97 genes may be observed in patients receiving dosing regimen of about ≧100 mg QW. Further, HDP-CDV treatment may reduce the incidence of BKV events, despite a high incidence of BK viruria in subjects (54%) at the time of enrollment, persisting during the active treatment period.

In some embodiments, the compound of the current invention may be administered orally with food. Food may be consumed before, concurrently, or after administration of the compound or a pharmaceutically acceptable salt and/or composition thereof. Food may be consumed about 30′, 60′, 90′, 120′, 150′, 180′, 210′, 240′, or >270′ before or after the administration of the compound.

### Prevention of VZV Spread

HDP-CDV suspensions of the current embodiments may be effective in preventing VZV spread. The HDP-CDV dose for prevention of VZV spread may be <1.00 mg/kg, about 1.25 mg/kg, about 2.5 mg/kg, about 5.0 mg/kg, or <25 mg/kg. A single dose of 1-20 mg/kg, 2-20 mg/kg, 3-20 mg/kg, 4-20 mg/kg, 5-20 mg/kg, 6-20 mg/kg, 7-20 mg/kg, 8-20 mg/kg, 9-20 mg/kg, or 10-20 mg/kg given on day −1, 2, 3, 4, may be effective in preventing VZV spread for 5 or more days. In one embodiment, a single dose of 20 mg/kg given on day 1, 2, or 4 may be effective in preventing VZV spread for 5 or more days.

The long intracellular half-life of the active antiviral anabolite of HDP-CDV may provide long-lasting antiviral activity, which may greatly increase the therapeutic potential for serious zoster infections.

The present methods find use in treating, i.e., reducing, relieving, ameliorating, preventing or inhibiting neuropathic pain in a subject or patient in need thereof. The patient may be subject to suffering neuropathic pain chronically or intermittently. The patient may or may not be exhibiting or experiencing symptoms of neuropathic pain at the time of treatment. The neuropathic pain may be centrally or peripherally mediated.

Neuropathic pain results from pathology in the nervous system. Notable features of neuropathic pain include (1) widespread pain not otherwise explainable; (2) evidence of sensory deficit; (3) burning pain; (4) pain to light stroking of the skin (allodynia); and (5) enhanced stimulus-dependent pain (hyperalgesia) and (6) attacks of pain without seeming provocation (stimulus-independent pain). Mechanisms of neuropathic pain are described, for example, in Zhuo, Molecular Pain (2007) 3:14; Campbell and Meyer, Neuron (2006) 52(1):77-92; Dworkin, et al., Arch Neurol (2003) 60:1524-34.

Neuropathic pain originates from a lesion of the nervous system. Any of a number of disease conditions or injuries can be the underlying cause of neuropathic pain. For example, the patient may be suffering from a metabolic disease (e.g., diabetic neuropathy), an autoimmune disease (e.g., multiple sclerosis), a viral infection (e.g., shingles and sequelae, postherpetic neuralgia), vascular disease (e.g. stroke), trauma and/or cancer. See, e.g. Campbell and Meyer, Neuron (2006) 52(1):77-92; Dworkin et al., Arch Neurol (2003) 60; 1524-34.

In some embodiments, the patient is suffering from neuropathic pain, for example, as a result of post-herpetic neuralgia. In some embodiments, the patient is suffering from neuropathic pain, for example, as a result of VZV infection.

HDP-CDV may be used to treat difficult zoster infections and prevent post-herpetic neuralgia. The current embodiments provide prevention of outbreak of shingles with the compounds of Formula I or II. The compounds of Formula I or II may be effective in ameliorating pain associated with outbreak of shingles and with postherpetic neuralgia. The compounds of Formula I or II may be co-administered or administered sequentially with pain relievers such as NSAIDs and/or with drugs commonly used to treat depression (antidepressants). The combination of a compound of Formula I or Formula II and an antidepressant may be effective in reducing paid and promote sleep in patients of shingles and/or postherpetic neuralgia. The subjects taking Formula I or II for ameliorating pain associated with postherpetic neuralgia may or may not have been vaccinated with a herpes zoster vaccine. In one embodiment, a single dose of 1-20 mg/kg of Formula I or II given on day 1, 2, 3, 4, 5, 6, 7, 8, 9, 10, or up to day 20 may be effective in reducing symptoms, e.g., pain, associated with shingles and/or postherpetic neuralgia.

### Compounds

In one embodiment, the antiviral compound is, e.g., cidofovir covalently linked to a lipid. In one embodiment, the antiviral compound has the structure:

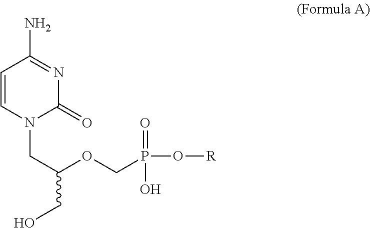

wherein R is H; optionally substituted alkyl, e.g., C₁₋₃₀ alkyl; alkenyl, e.g., C₂₋₃₀ alkenyl; or alkynyl, e.g., C₂₋₃₀ alkynyl; acyl; mono- or di-phosphate; alkylglycerol, alkylpropanediol, 1-S-alkylthioglycerol, alkoxyalkanol or alkylethanediol. In one embodiment R is an alkoxyalkanol. For example, R is —(CH₂)ₘ—O—(CH₂)ₙ—CH₃ wherein, e.g., m is 1-5 and n is 1-25; or m is 2-4 and n is 10-25.

In another embodiment, the antiviral compound has the following structure:

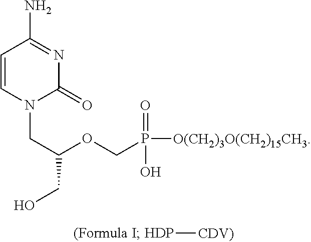

In one embodiment, the antiviral compound may be derived by deamination or hydrolysis of HDP-CDV and may have the structure:

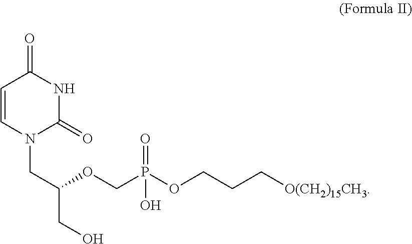

HDP-CDV is an orally administered lipid conjugate of the synthetic nucleotide analog CDV, which is absorbed in the small intestine and delivered to target organs throughout the body. Inside the cell, HDP-CDV is cleaved to release CDV, presumably by phospholipases, which is converted to the active antiviral agent, CDV-diphosphate (CDV-PP), by intracellular anabolic kinases. This active metabolite of HDP-CDV has been shown to have a long intracellular half-life of approximately 6.5 days.

In one embodiment, the antiviral compound is a derivative of adefovir and has the structure:

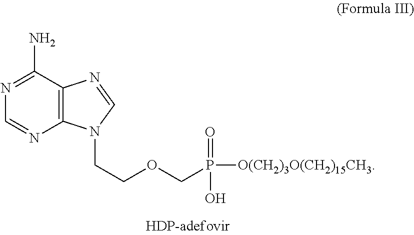

Lipid conjugates or derivatives of compounds, including of the following agents, also may be used: analgesic; anesthetic; anorectic; anti-adrenergic; anti-allergic; anti-anginal; anti-anxiety; anti-arthritic; anti-asthmatic; anti-atherosclerotic; antibacterial; anticoagulant; anticonvulsant; antidepressant; antidiabetic; antidiarrheal; antidiuretic; anti-estrogen; antifibrinolytic; antifungal; antiglaucoma agent; antihistamine; anti-infective; anti-inflammatory; antikeratinizing agent; antimalarial; antimicrobial; antimigraine; antimitotic; antimycotic, antinauseant, antineoplastic, antineutropenic, antiobessional agent; antiparasitic; antiparkinsonian; antiperistaltic, antipneumocystic; antiproliferative; liver disorder treatment; psychotropic; serotonin inhibitor; serotonin receptor antagonist; steroid; stimulant; suppressant; thyroid hormone; thyroid inhibitor; thyromimetic; tranquilizer; agent for treatment of amyotrophic lateral sclerosis; agent for treatment of cerebral ischemia; agent for treatment of Paget's disease; agent for treatment of unstable angina; uricosuric; vasoconstrictor; vasodilator; vulnerary; or a wound healing agent.

### Anti-Viral Activity

HDP-cidofovir (HDP-CDV) has broad spectrum in vitro antiviral activity against all dsDNA viruses of importance to immunocompromised patients, including transplant recipients and has not been associated with either myelosuppression or nephrotoxicity in extensive clinical trials.

In one embodiment, a therapeutically effective dosage to treat a virus infection, for example, an BKV infection may produce a serum concentration of anti-viral agent of about 0.1 ng/ml to about 50-100 μg/ml. The pharmaceutical compositions, in another embodiment, should provide a dosage of from about 0.001 mg to about 2000 mg of compound per kilogram of body weight per day. Pharmaceutical dosage unit forms are prepared, e.g., to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The in vitro antiviral activity of relevant, currently available nucleoside/nucleotide analogs against AdV, BKV, EBV, herpes simplex virus 1 (HSV-1), VZV and human herpesvirus 6 (HHV-6), assessed in cell culture experiments, is presented in Table 1.

Cell culture experiments have demonstrated >100 to 1000-fold higher potency of HDP-CDV in comparison to cidofovir, ganciclovir and other nucleoside analogs against dsDNA viruses of interest, e.g., adenovirus (AdV) or BK virus (BKV).

TABLE 1
Comparison of in vitro Antiviral Activity
of Nucleoside/Nucleotide Analogs

| | EC50s (μM) | | | | |
|---|---|---|---|---|---|
| | HDP- | | | | |
| Virus | CDV | Cidofovir | Ganciclovir | Maribavir | Acyclovir |
| CMV | 0.001 | 0.4 | 3.8 | 0.31 | >200 |
| VZV | 0.0004 | 0.5 | 1.3 | No Activity | 3.6 |
| HHV-6(B) | 0.007 | 5.4 | 5.9 | ND | 100 |
| AdV | 0.02 | 1.3 | 4.5-33 | ND | >100 |
| EBV | 0.04 | >108 | 0.9 | 0.63 | 8.5 |
| HSV-1 | 0.06 | 5.5 | 0.007 | No Activity | 2.5 |
| BKV | 0.13 | 115 | >200 | ND | >200 |

Methods of treating, preventing, or ameliorating disorders such as viral infections are provided herein. In practicing the methods, effective amounts of a derivative, e.g. of an anti-viral compound, may be administered in any desired manner, e.g., via oral, rectal, nasal, topical (including buccal and sublingual), vaginal, or parenteral (including subcutaneous, intramuscular, subcutaneous, intravenous, intradermal, intraocular, intratracheal, intracisternal, intraperitoneal, and epidural) administration.

### Combination Therapy

The compounds or compositions provided herein may also be used in combination with an enhancer agent, with other active ingredients, or with an immunosuppressant agent. In certain embodiments, the compounds may be administered in combination, or sequentially, with another therapeutic agent or an enhancer. Such other therapeutic agents include those known for treatment, prevention, or amelioration of one or more symptoms associated with viral infections. It should be understood that any suitable combination of the compounds provided herein with one or more of the above-mentioned compounds and optionally one or more further pharmacologically active substances are considered to be within the scope of the present disclosure. In another embodiment, the compound provided herein is administered prior to or subsequent to the one or more additional active ingredients. In one embodiment, two or more of the antiviral agents disclosed herein are administered serially or in combination.

The amount of some enhancers can be selected using methods known in the art to enhance the bioavailability of the anti-viral agent. Any amount can be used that provides a desired response by some enhancers. The dosages may range, in a non-limiting example, from 0.001 mg to about 2000 mg of compound per kilogram of body weight per day, e.g., 0.01 to 500 mg/kg, or e.g., 0.1-20 mg/kg.

The co-administration of the compound or compositions provided herein with another agent may have a synergistic effect in treating BKV infection, reactivation of BKV, or preventing end organ damage or impairment in a subject infected with BKV. Specific examples of such combinations include, but are not limited to: HDP-CDV or compound of Formula II, or a pharmaceutically acceptable salt thereof in combination with at least one immunosuppressant agents. Exemplary immunosurpressant agent include, but are not limited to, Daclizumab, Basiliximab, Tacrolimus, Sirolimus, Mycophenolate (as sodium or mofetil), Cyclosporine A, Glucocorticoids, Anti-CD3 monoclonal antibodies (OKT3), Antithymocyte globulin (ATG), Anti-CD52 monoclonal antibodies (campath 1-H), Azathioprine, Everolimus, Dactinomycin, Cyclophosphamide, Platinum, Nitrosurea, Methotrexate, Azathioprine, Mercaptopurine, Muromonab, IFN gamma, Infliximab, Etanercept, Adalimumab, Tysabri (Natalizumab), Fingolimodm and a combination thereof. In some embodiments, the pharmaceutical composition includes, e.g., HDP-CDV, Tysabri (natalizumab), and a pharmaceutically acceptable carrier.

In one embodiment, the pharmaceutical composition described herein comprises, e.g., HDP-CDV, or pharmaceutically acceptable salt thereof and one or more medication for treating viral infection, e.g., polyomavirus JC virus (“JCV”), that causes Progressive multifocal leukoencephalopathy (“PML”), in at least one pharmaceutically acceptable carrier. In one embodiment, one or more medication is selected from the group consisting of RITUXAN® (rituximab), RAPTIVA® (efalizumab), TYSABRI® (natalizumab), MYFORTIC® (mycophenolic acid), AVONEX® (interferon beta-1a), REMICADE® (infliximab), ENBREL® (etanercept), HUMIRA® (adalimumab), CELLCEPT® (mycophenolate mofetil), and a combination thereof in at least one pharmaceutically acceptable carrier.

In another embodiment, the pharmaceutical composition described herein includes HDP-CDV or compound of Formula II or a pharmaceutically acceptable salt thereof, in at least one pharmaceutically acceptable carrier.

### Pharmaceutical Compositions

Pharmaceutical carriers suitable for administration of the compounds provided herein include any such carriers known to those skilled in the art to be suitable for the particular mode of administration. The compounds may be formulated as the sole pharmaceutically active ingredient in the composition or may be combined with other active ingredients.

Liquid form preparations include solutions, suspensions, and emulsions, for example, water or water/propylene glycol solutions. For parenteral injection, liquid preparations can be formulated in solution in aqueous polyethylene glycol solution. Transdermal administration can be performed using suitable carriers.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation may be in unit dosage form. In such form the preparation may be subdivided into unit doses containing appropriate quantities of the active component. The unit dosage form may be a packaged preparation, the package containing discrete quantities of preparation, such as packeted tablets, capsules, and powders in vials or ampoules. Also, the unit dosage form may be a capsule, tablet, cachet, or lozenge itself, or it can be the appropriate number of any of these in packaged form.

Compounds of Formula I and II free acid tablets (20 mg-200 mg) may be formulated as dry-blend, direct-compressed tablet containing 20-200 mg HDP-CDV or Formula II active ingredient. In addition to the active ingredient, HDP-CDV or Formula II tablets may contain microcrystalline cellulose, mannitol, crospovidone and magnesium stearate. The tablets of the current invention may contain 20-40 mg, 50 mg, 75 mg, 100 mg, 150 mg, or 200 mg of the compound of Formula I or Formula II.

Tablets of various strengths may be developed. The tablets may be compressed from a common blend, while varying the drug load for different strengths. The 20-40 mg, 50 mg, 75 mg, 100 mg, 150 mg, or 200 mg dosage forms, may be round, biconvex tablets with dimensions 7.3 mm×3.5 mm, 7.9 mm×3.8 mm, or 10.5 mm×4.4 mm. HDP-CDV as the free acid may be formulated as direct compression, instant release tablets containing 20, 50, 75, 100, or 200 mg HDP-CDV. The tablet may comprise one or more pharmaceutically acceptable excipients, for example, diluent, binder or flow aid, e.g., silicified microcrystalline cellulose, mannitol, microcrystalline cellulose; disintegrant, e.g., crospovidone; and lubricant, e.g., magnesium stearate.

Tablets comprising 50-200 mg HDP-CDV or the compound of Formula II may be prepared in combination with varying concentrations of one or more pharmaceutically acceptable excipients, for example, diluent, binder or flow aid, e.g., silicified microcrystalline cellulose (about, e.g., 22% wt/wt), mannitol (about, e.g., 34% wt/wt), microcrystalline cellulose (about, e.g., 11% wt/wt); disintegrant, e.g., crospovidone (about, e.g., 3-4% wt/wt); and lubricant, e.g., magnesium stearate (about, e.g., 0.7-0.9% wt/wt).

The term “unit dosage form,” as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the novel unit dosage forms of this invention are dictated by and directly dependent on (a) the unique characteristics of the active material and the particular effect to be achieved and (b) the limitations inherent in the art of compounding such an active material for use in humans and animals, as disclosed in detail in this specification, these being features of the present invention.

An embodiment of the current invention provides a suspension of compound of Formula I or Formula II in a viscosity modifier or thickener. For example, HDP-CDV may be in a suspension with carboxymethylcellulose, methylcellulose, hydroxyethyl methylcellulose, ethylcellulose, hydroxyethyl cellulose, poly(ethylene) oxide, hydroxypropyl methylcellulose, and/or nitrocellulose.

Compositions comprising the compounds disclosed herein may be suitable for oral, rectal, nasal, topical (including buccal and sublingual), vaginal, or parenteral (including subcutaneous, intramuscular, subcutaneous, intravenous, intradermal, intraocular, intratracheal, intracistemal, intraperitoneal, and epidural) administration.

The compositions may conveniently be presented in unit dosage form and may be prepared by conventional pharmaceutical techniques. Such techniques include the step of bringing into association one or more compositions provided herein and one or more pharmaceutical carriers or excipients.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof may be mixed with one or more suitable pharmaceutical carriers. The compounds may be derivatized as the corresponding salts, esters, enol ethers or esters, acetals, ketals, orthoesters, hemiacetals, hemiketals, acids, bases, solvates, hydrates or derivatives prior to formulation. The concentrations of the compounds in the compositions are effective for delivery of an amount, upon administration, that treats, prevents, or ameliorates one or more of the symptoms of the target disease or disorder. In one embodiment, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

Compositions suitable for oral administration may be presented as discrete units such as, but not limited to, tablets, caplets, pills or dragees capsules, or cachets, each containing a predetermined amount of one or more of the compositions; as a powder or granules; as a solution or a suspension in an aqueous liquid or a non-aqueous liquid; or as an oil-in-water liquid emulsion or a water-in-oil emulsion or as a bolus, etc.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents, preservatives, flavoring agents, and the like, for example, acetate, sodium citrate, cyclodextrine derivatives, sorbitan monolaurate, triethanolamine sodium acetate, triethanolamine oleate, and other such agents. Methods of preparing such dosage forms are known, or will be apparent, to those skilled in this art; for example, see Remington's Pharmaceutical Sciences, Mack Publishing Company, Easton, Pa., 15th Edition, 1975.

Compositions of the present invention suitable for topical administration in the mouth include for example, lozenges, having the ingredients in a flavored basis, usually sucrose and acacia or tragacanth; pastilles, having one or more of the compositions of the present invention in an inert basis such as gelatin and glycerin, or sucrose and acacia; and mouthwashes, having one or more of the compositions of the present invention administered in a suitable liquid carrier.

The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

Compositions suitable for topical administration to the skin may be presented as ointments, creams, gels, and pastes, having one or more of the compositions administered in a pharmaceutical acceptable carrier.

Compositions for rectal administration may be presented as a suppository with a suitable base comprising, for example, cocoa butter or a salicylate.

Compositions suitable for nasal administration, when the carrier is a solid, include a coarse powder having a particle size, for example, in the range of 20 to 500 microns which is administered in the manner in which snuff is taken, (i.e., by rapid inhalation through the nasal passage from a container of the powder held close up to the nose). When the carrier is a liquid (for example, a nasal spray or as nasal drops), one or more of the compositions can be admixed in an aqueous or oily solution, and inhaled or sprayed into the nasal passage.

Compositions suitable for vaginal administration may be presented as pessaries, tampons, creams, gels, pastes, foams or spray formulations containing one or more of the compositions and appropriate carriers.

Compositions suitable for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. The compositions may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules, and tablets of the kind previously described above.

Pharmaceutical organic or inorganic solid or liquid carrier media suitable for enteral or parenteral administration can be used to fabricate the compositions. Gelatin, lactose, starch, magnesium stearate, talc, vegetable and animal fats and oils, gum, polyalkylene glycol, water, or other known carriers may all be suitable as carrier media.

Compositions may be used as the active ingredient in combination with one or more pharmaceutically acceptable carrier mediums and/or excipients. As used herein, “pharmaceutically acceptable carrier” includes any and all carriers, solvents, diluents, or other liquid vehicles, dispersion or suspension aids, surface active agents, isotonic agents, thickening or emulsifying agents, preservatives, solid binders, lubricants, adjuvants, vehicles, delivery systems, disintegrants, absorbents, preservatives, surfactants, colorants, flavorants, or sweeteners and the like, as suited to the particular dosage form desired.

Additionally, the compositions may be combined with pharmaceutically acceptable excipients, and, optionally, sustained-release matrices, such as biodegradable polymers, to form therapeutic compositions. A “pharmaceutically acceptable excipient” includes a non-toxic solid, semi-solid or liquid filler, diluent, encapsulating material or formulation auxiliary of any type.

It will be understood, however, that the total daily usage of the compositions will be decided by the attending physician within the scope of sound medical judgment. The specific therapeutically effective dose level for any particular host will depend upon a variety of factors, including for example, the disorder being treated and the severity of the disorder; activity of the specific composition employed; the specific composition employed, the age, body weight, general health, sex and diet of the patient; the time of administration; route of administration; rate of excretion of the specific compound employed; the duration of the treatment; drugs used in combination or co-incidental with the specific composition employed; and like factors well known in the medical arts. For example, it is well within the skill of the art to start doses of the composition at levels lower than those required to achieve the desired therapeutic effect and to gradually increase the dosage until the desired effect is achieved.

Compositions are preferably formulated in dosage unit form for ease of administration and uniformity of dosage. “Dosage unit form” as used herein refers to a physically discrete unit of the composition appropriate for the host to be treated. Each dosage should contain the quantity of composition calculated to produce the desired therapeutic affect either as such, or in association with the selected pharmaceutical carrier medium.

Exemplary unit dosage formulations are those containing a daily dose or unit, daily sub-dose, or an appropriate fraction thereof, of the administered ingredient. The dosage will depend on host factors such as weight, age, surface area, metabolism, tissue distribution, absorption rate and excretion rate. Exemplary systemic dosages for all of the herein described conditions are those ranging from 0.01 mg/kg to 2000 mg/kg of body weight per day as a single daily dose or divided daily doses. Typical dosages for topical application are those ranging from 0.001 to 100% by weight of the active compound.

The therapeutically effective dose level will depend on many factors as noted above. In addition, it is well within the skill of the art to start doses of the composition at relatively low levels, and increase the dosage until the desired effect is achieved.

Compositions comprising a compound disclosed herein may be used with a sustained-release matrix, which can be made of materials, usually polymers, which are degradable by enzymatic or acid-based hydrolysis or by dissolution. Once inserted into the body, the matrix is acted upon by enzymes and body fluids. A sustained-release matrix for example is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (copolymers of lactic acid and glycolic acid).

The compounds may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The liposome can contain, in addition to one or more compositions of the present invention, stabilizers, preservatives, excipients, and the like. Examples of lipids are the phospholipids and the phosphatidyl cholines (lecithins), both natural and synthetic. Methods to form liposomes are known in the art.

The compounds may be formulated as aerosols for application, such as by inhalation. These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

Compositions comprising the compounds disclosed herein may be used in combination with other compositions and/or procedures for the treatment of the conditions described above.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

In therapeutic applications, the dosages of the pharmaceutical compositions used in accordance with the invention vary depending on the agent, the age, weight, and clinical condition of the recipient patient, and the experience and judgment of the clinician or practitioner administering the therapy, among other factors affecting the selected dosage. Dosages can range from about 0.01 mg/kg to about 200 mg/kg. In preferred aspects, dosages can range from about 0.1 mg/kg to about 10 mg/kg. In an aspect, the dose will be in the range of about 1 mg to about 1 g; about 10 mg to about 500 mg; about 20 mg to about 400 mg; about 40 mg to about 400 mg; or about 50 mg to about 400 mg, in single, divided, or continuous doses (which dose may be adjusted for the patient's weight in kg, body surface area in m², and age in years). In certain embodiments, the amount per dosage form can be about 0.1 mg to about 1000 mg, e.g., about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 95 or 100 mg, 150 mg, 200 mg, or more.

In another embodiment, the invention provides compositions (e.g., pharmaceutical compositions) with desirable pharmacokinetic characteristics. For example, the compositions of the invention may provide a blood level of the compound of Formula I or Formula II, which, after metabolism to the therapeutically-active form (i.e., cidofovir), results in blood levels of the metabolite that do not induce toxicity (e.g., nephrotoxicity).

An effective amount of a pharmaceutical agent is that which provides an objectively identifiable improvement as noted by the clinician or other qualified observer. As used herein, the term “dosage effective manner” refers to amount of an active compound to produce the desired biological effect in a subject or cell.

In another embodiment, HDP-CDV or compound of Formula II of the present invention can be administered to a subject as a single dose. In another embodiment, HDP-CDV or compound of Formula II of the present invention can be administered to a subject in multiple doses. Multiple doses can be administered regularly, for example, once every 12 hours, once a day, every 2 days, every 3 days, every 4 days, every 5 days, every 6 days, every 7 days, every 8 days, every 9 days, every 10 days, every 11 days, every 12 days, every 13 days, every 14 days or every 15 days. For example, doses can be administered twice per week. Moreover, each individual dose can be administered with the same or a different dosage.

For example, a subject can be administered with a first dose of 2 mg/kg of compound of Formula I or Formula II followed by one or more additional doses at 2 mg/kg. For example, a subject can be administered with a first dose of 2 mg/kg followed by one or more additional doses at 1 mg/kg. For example, a subject can be administered with a first dose of 2 mg/kg followed by one or more additional doses at 3 mg/kg. For example, a subject can be administered with a first dose of 4 mg/kg followed by one or more additional doses at 4 mg/kg.

Multiple doses can also be administered at variable time intervals. For example, the first 2, 3, 4, 5, 6, 7, or 8 or more doses can be administered at an interval of 6 days followed by additional doses administered at an interval of 7 days. For example, the first 2, 3, 4, 5, 6, 7, or 8 or more doses can be administered at an interval of 7 days followed by additional doses administered at an interval of 3 days.

In another embodiment, the invention provides an oral dosage form comprising a compound of Formula I for the therapeutic and/or prophylactic treatment of viral infection in a subject, wherein said oral dosage form, upon administration to a human at a dosage of 2 mg/kg of said compound, provides an AUC_{0-inf} of said compound of about 2000 to about 4000 h*ng/mL, e.g., about 2500 to about 3000 h*ng/mL. In some embodiments, the AUC_{0-inf} of said compound is about 2000, 2100, 2200, 2300, 2400, 2500, 2600, 2700, 2800, 2900, 3000, 3100, 3200, 3300, 3400, 3500, 3600, 3700, 3800, 3900, or 4000 h*ng/mL or any range therein. AUC_{0-inf} can be determined by any of the well-known methods in the art and as described in the examples herein.

In another embodiment, the invention provides an oral dosage form comprising a compound of Formula I or Formula II for the therapeutic and/or prophylactic treatment of viral infection in a subject, wherein said oral dosage form, upon administration to a human at a dosage of 2 mg/kg of said compound, provides a Cₘₐₓ of said compound of about 100 to about 500 ng/mL, e.g., about 200 to about 400 ng/mL. In some embodiments, the Cₘₐₓ of the compound is about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ng/mL or any range therein. Cₘₐₓ can be determined by any of the well-known methods in the art and as described in the examples herein.

In another embodiment, the invention provides an oral dosage form comprising a compound of Formula I or Formula II for the therapeutic and/or prophylactic treatment of viral infection in a subject, wherein said oral dosage form, upon administration to a human at a dosage of 2 mg/kg of said compound of Formula I or Formula II and metabolism of said compound of Formula I or Formula II to cidofovir, provides a Cₘₐₓ of said cidofovir that is less than about 30% of the Cₘₐₓ of said compound of Formula I or Formula II, e.g., less that about 20% of the Cₘₐₓ of said compound of Formula I or Formula II. In some embodiments, the Cₘₐₓ of the metabolite (i.e., cidofovir) is less than about 50%, 45%, 40%, 35%, 30%, 25%, 20%, 15%, or 10% of the Cₘₐₓ of the compound of Formula I.

In another embodiment, the invention provides an oral dosage form comprising a compound of Formula I or Formula II, wherein upon administration to a human at a dosage of 2 mg/kg of said compound of Formula I or Formula II, provides an AUC_{0-inf} of cidofovir of about 1000 to about 5000 h*ng/mL, e.g., about 1500 to about 4000 h*ng/mL. In some embodiments, the AUC_{0-inf} of cidofovir is about 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 h*ng/mL or any range therein.

In another embodiment, the invention provides an oral dosage form comprising a compound of Formula I or Formula II, wherein upon administration to a human at a dosage of 2 mg/kg of said compound of Formula I or Formula II, provides a Cₘₐₓ of cidofovir of about 10 to about 100 ng/mL, e.g., about 20 to about 70 ng/mL. In some embodiments, the Cₘₐₓ of the compound of Formula I or Formula II is about 10, 20, 30, 40, 50, 60, 70, 80, 90, or 100 ng/mL or any range therein.

In certain embodiments, the oral dosage form provides more than one of the pharmacokinetic characteristics described above, e.g., the AUC_{0-inf} or Cₘₐₓ of the compound of Formula I, Formula II, or the metabolite (i.e., cidofovir) or the Cₘₐₓ ratio of the metabolite (i.e., cidofovir) to the compound of Formula I or Formula II, e.g., 2, 3, 4, or more of the pharmacokinetic characteristics in any combination.

The pharmacokinetic behavior of a composition will vary somewhat from subject to subject within a population. The numbers described above for the compositions of the invention are based on the average behavior in a population. The present invention is intended to encompass compositions that on average fall within the disclosed ranges, even though it is understood that certain subjects may fall outside of the ranges.

The pharmaceutical compositions can be included in a container, pack, or dispenser together with instructions for administration.

The compounds of the present invention are capable of further forming salts. All of these forms are also contemplated within the scope of the claimed invention. It should be understood that all references to pharmaceutically acceptable salts include solvent addition forms (solvates) or crystal forms (polymorphs) as defined herein, of the same salt.

The compounds of the present invention can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

### Effect of Food

In some embodiments, the pharmaceutical composition of the current embodiments, e.g., tablet or suspension, may be provided to a subject when the subject is either fasted or in fed conditions. In one embodiment, the composition comprising Formula I or II compound may be provided to a subject having an empty stomach, e.g., after fasting for less than 24 hours but more than 12 hours, more than 11 hours, more than 10 hours, more than 8 hours, or more than 5 hours.

In other embodiments, the composition comprising Formula I or II compound may be provided to a subject in combination with food or subsequent to having food. In one embodiment, compound of Formula I or Formula II may be taken by a subject on an empty stomach.

### Patient Population

In certain embodiments, HDP-CDV or the compound of Formula II (“Compound”), a composition comprising a Compound, or a combination therapy is administered to a mammal which is about 1 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old.

In certain embodiments, a Compound, a composition comprising a Compound, or a combination therapy is administered to a human at risk for a virus infection. In certain embodiments, a Compound, a composition comprising a Compound, or a combination therapy is administered to a human with a virus infection. In certain embodiments, the patient is a human about 1 to 6 months old, 6 to 12 months old, 1 to 5 years old, 5 to 10 years old, 5 to 12 years old, 10 to 15 years old, 15 to 20 years old, 13 to 19 years old, 20 to 25 years old, 25 to 30 years old, 20 to 65 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old.

In some embodiments, a Compound, a composition comprising a Compound, or a combination therapy is administered to a human infant. In other embodiments, a Compound, or a combination therapy is administered to a human child. In other embodiments, a Compound, a composition comprising a Compound, or a combination therapy is administered to a human adult. In yet other embodiments, a Compound, a composition comprising a Compound, or a combination therapy is administered to an elderly human.

### DEFINITIONS

As used herein, the terms “subject” and “patient” are used interchangeably. As used herein, the term “subject” or “subjects” refer to an animal (e.g., birds, reptiles, and mammals), a mammal including a non-primate (e.g., a camel, donkey, zebra, cow, pig, horse, goat, sheep, cat, dog, rat, and mouse), and a primate (e.g., a monkey, chimpanzee, and a human).

The term “pharmaceutically acceptable salt” as used herein, unless otherwise specified, includes those salts which are, within the scope of sound medical judgment, suitable for use in contact with the tissues of hosts without undue toxicity, irritation, allergic response and the like, and are commensurate with a reasonable benefit/risk ratio and effective for their intended use. The salts can be prepared in situ during the final isolation and purification of one or more compounds of the composition, or separately by reacting the free base function with a suitable organic acid. Non-pharmaceutically acceptable acids and bases also find use herein, as for example, in the synthesis and/or purification of the compounds of interest. Non-limiting examples of such salts are (a) acid addition salts formed with inorganic salts (for example hydrochloric acid, hydrobromic acid, sulfuric acid, phosphoric acid, nitric acid, and the like), and salts formed with organic salts such as acetic acid, oxalic acid, tartaric acid, succinic acid, ascorbic acid, benzoic acid, tannic acid, and the like; (b) base addition salts formed with metal cations such as zinc, calcium, magnesium, aluminum, sodium, potassium, copper, nickel and the like; (c) combinations of (a) and (b). Also included as “pharmaceutically acceptable salts” are amine salts.

For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present invention also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, diethylamine, diethylaminoethanol, ethylenediamine, imidazole, lysine, arginine, morpholine, 2-hydroxyethylmorpholine, dibenzylethylenediamine, trimethylamine, piperidine, pyrrolidine, benzylamine, tetramethylammonium hydroxide and the like.

The term “pharmaceutically acceptable esters” as used herein, unless otherwise specified, includes those esters of one or more compounds, which are, within the scope of sound medical judgment, suitable for use in contact with the tissues of hosts without undue toxicity, irritation, allergic response and the like, are commensurate with a reasonable benefit/risk ratio, and are effective for their intended use.

The term “pharmaceutically acceptable derivative” includes a compound that is metabolized, for example, hydrolyzed or oxidized, in the host to form an active compound. Typical examples of derivatives include compounds that have biologically labile protecting groups on a functional moiety of the active compound. Derivatives include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

The term “bioenhancer(s)” refers to agents that enhance the availability of biological agent(s) through one or more mechanism(s) in warm blooded animals comprising increasing the bioavailability, enhancing the serum concentration, improving gastrointestinal absorption, improving systemic utilization, improving cross over through certain biological barriers such as respiratory lining, urinary lining, blood brain barrier and skin. The bioenhancer may be one or more ingredients selected from phytochemicals, Nootropic agents, anti obese agents, antiinflammatory agents, anti cholesterol agents, anti arthritic agents, anti diabetic agents, anti microbial agents, anti fungal agents, anti cancer agents, anti hypertensive agents, analgesic agents, anti platelet aggregation agents, anti atherosclerotic agents, antioxidants, anti thrombotic agents, antibiotic agents, anti malarial agents, anti osteoporotic agents, probiotics agents, anti fungal agents, immune potentiating agents, anti viral agents, anti histamines, muscle relaxants, anti depressants, hypnotic agents and their salts thereof.

The term “effective amount” includes an amount required for prevention, treatment, or amelioration of one or more of the symptoms of diseases or disorders provided herein. As used herein, the term “effective amount” in the context of administering a therapy to a subject refers to the amount of a therapy which is sufficient to achieve one, two, three, four, or more of the following effects: (i) reduce or ameliorate the severity of a viral infection or a symptom associated therewith; (ii) reduce the duration of a viral infection or a symptom associated therewith; (iii) prevent the progression of a viral infection or a symptom associated therewith; (iv) cause regression of a viral infection or a symptom associated therewith; (v) prevent the development or onset of a viral infection or a symptom associated therewith; (vi) prevent the recurrence of a viral infection or a symptom associated therewith; (vii) reduce or prevent the spread of a virus from one cell to another cell, or one tissue to another tissue; (ix) prevent or reduce the spread of a virus from one subject to another subject; (x) reduce organ failure associated with a viral infection; (xi) reduce hospitalization of a subject; (xii) reduce hospitalization length; (xiii) increase the survival of a subject with a viral infection; (xiv) eliminate a virus infection; and/or (xv) enhance or improve the prophylactic or therapeutic effect(s) of another therapy.

As used herein, the term “in combination,” in the context of the administration of two or more therapies to a subject, refers to the use of more than one therapy (e.g., more than one prophylactic agent and/or therapeutic agent). The use of the term “in combination” does not restrict the order in which therapies are administered to a subject with a viral infection. A first therapy (e.g., a first prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject with a viral infection.

As used herein, the term “infection” means the invasion by, multiplication and/or presence of a virus in a cell or a subject. In one embodiment, an infection is an “active” infection, i.e., one in which the virus is replicating in a cell or a subject. Such an infection is characterized by the spread of the virus to other cells, tissues, and/or organs, from the cells, tissues, and/or organs initially infected by the virus. An infection may also be a latent infection, i.e., one in which the virus is not replicating. In one embodiment, an infection refers to the pathological state resulting from the presence of the virus in a cell or a subject, or by the invasion of a cell or subject by the virus.

As used herein, the terms “prevent,” “preventing” and “prevention” in the context of the administration of a therapy(ies) to a subject to prevent a viral infection refer to one or more of the following effects resulting from the administration of a therapy or a combination of therapies: (i) the inhibition of the development or onset of a viral infection and/or a symptom associated therewith; (ii) the inhibition of the recurrence of a viral infection and/or a symptom associated therewith; (iii) the inhibition of en-organ damage or impairment in a subject infected with a virus, e.g., BKV.

As used herein, the terms “prophylactic agent” and “prophylactic agents” refer to any agent(s) which can be used in the prevention of a viral infection or a symptom associated therewith. Preferably, a prophylactic agent is an agent which is known to be useful to or has been or is currently being used to prevent or impede the onset, development, progression and/or severity of a viral infection or a symptom associated therewith.

As used herein, the term “prophylactically effective amount” refers to the amount of a therapy (e.g., prophylactic agent) which is sufficient to prevent a viral infection or a symptom thereof in a subject.

As used herein, the term “synergistic,” in the context of the effect of therapies, refers to a combination of therapies which is more effective than the additive effects of any two or more single therapies. In a specific embodiment, a synergistic effect of a combination of therapies permits the use of lower dosages of one or more of therapies and/or less frequent administration of said therapies to a subject with a viral infection. In certain embodiments, the ability to utilize lower dosages of therapies (e.g., prophylactic or therapeutic agents) and/or to administer said therapies less frequently reduces the toxicity associated with the administration of said therapies to a subject without reducing the efficacy of said therapies in the prevention or treatment of a viral infection. In some embodiments, a synergistic effect results in improved efficacy of therapies (e.g., prophylactic or therapeutic agents) in the prevention, management and/or treatment of a viral infection. In some embodiments, a synergistic effect of a combination of therapies (e.g., prophylactic or therapeutic agents) avoids or reduces adverse or unwanted side effects associated with the use of any single therapy.

As used herein, the term “therapeutically effective amount” refers to the amount of a therapy, which is sufficient to treat and/or manage a viral infection. As used herein, the terms “therapeutic agent” and “therapeutic agents” refer to any agent(s) which can be used in the prevention, treatment and/or management of a viral infection or a symptom associated therewith. Preferably, a therapeutic agent is an agent, which is known to be useful for, or has been or is currently being used for the prevention, treatment, and/or management of a viral infection or a symptom associated therewith.

As used herein, the terms “treat,” “treatment,” and “treating” refer in the context of administration of a therapy(ies) to a subject to treat a viral infection refer to one, two, three, four, five or more of the following effects resulting from the administration of a therapy or a combination of therapies: (i) the reduction or amelioration of the severity of a viral infection and/or a symptom associated therewith; (ii) the reduction in the duration of a viral infection and/or a symptom associated therewith; (iii) the regression of a viral infection and/or a symptom associated therewith; (iv) the reduction of the titer of a virus; (v) the reduction in organ failure associated with a viral infection; (vi) the reduction in hospitalization of a subject; (vii) the reduction in hospitalization length; (viii) the increase in the survival of a subject; (ix) the elimination of a virus infection; (x) the inhibition of the progression of a viral infection and/or a symptom associated therewith; (xi) the prevention of the spread of a virus from a cell, tissue or subject to another cell, tissue or subject; and/or (xii) the enhancement or improvement the therapeutic effect of another therapy.

The compounds provided herein may be enantiomerically pure, or be stereoisomeric or diastereomeric mixtures. It is understood that the disclosure of a compound herein encompasses any racemic, optically active, polymorphic, or steroisomeric form, or mixtures thereof, which preferably possesses the useful properties described herein, it being well known in the art how to prepare optically active forms and how to determine activity using the standard tests described herein, or using other similar tests which are well known in the art.

The invention will be further understood from the following non-limiting examples.

### EXAMPLES

### Example 1

### Clinical Studies

A HDP-CDV-201, a 9-11 week randomized, placebo-controlled, double-blind, dose-escalation clinical study (40 mg weekly [QW], 100 mg QW, 200 mg QW, 200 mg twice-weekly [BIW], and 100 mg BIW) of HDP-CDV for the prevention of BKV infection post-HCT was performed. Treatment was initiated at the time of engraftment and continued until Week 13 post-HCT.

HDP-CDV Dose/Regimen for Patients with Viral Infection

HDP-CDV was administered to patients suffering from one or more viral infections. Table 2 provides HDP-CDV doses and dosage regimens for patients suffering from one or more viral infections. Doses of HDP-CDV ranging from 1-4 mg/kg once or twice weekly were administered for up to 13 weeks.

TABLE 2
Doses and Dosage Regimens of HDP-CDV for Several Viral Infections

| | | HDP-CDV | Antiviral Medications | |
|---|---|---|---|---|
| Patient | Viral | Dose | (Previous (“Prev”)/ | |
| age, wt. | Infection | Regimen | Concomitant (“Con”)) | Virology Data |
| 63 yrs., | JCV | 2 mg/kg | Prev.: VALTREX ® | During treatment with HDP-CDV, the JCV |
| unknown | associated | BIW | Con.: VALTREX ® | viral load became undetectable (<300 |
| | PML | | | copies/mL) in the CSF and urine from an |
| | | | | initial level of 398 copies/mL in the CSF |
| | | | | and an initial level of 557 copies/mL in the urine. |
| 14 yrs., | ADV | 2 mg/kg | Prev.: CDV, FOS | No plasma adenovirus levels were |
| 58 kg | viremia | | Con.: FOS | available subsequent to the initial dose of |
| | BKV, CMV | | | HDP-CDV. |
| 29 yrs., | CMV, BKV | 2 mg/kg first | Prev.: ACV, leflunomide, GCV | CMV suppression continued during HDP- |
| 69 kg | hemorrhagic | dose; | | CDV therapy, after discontinuation of |
| | cystitis | 1 mg/kg | | ganciclovir. BK viruria decreased during |
| | | second dose; | | administration of HDP-CDV. |
| | | BIW | | |
| | | 2 mg/kg | | |
| | | thereafter | | |
| 21 yrs., | Molluscum | 2 mg/kg first | | No virology was done for this case. |
| 57.4 kg | contagiosum | dose; | | |
| | | 1 mg/kg | | |
| | | QW | | |
| | | thereafter | | |
| 43 yrs., | ADV | 2 mg/kg | Prev.: CDV, ACV, RBV | ADV viremia decreased from 430,000 |
| 70 kg | viremia | every three | | copies/mL to undetectable after 3 weeks |
| | | days | | (>3 log₁₀). Colon biopsy was negative for |
| | | | | ADV after HDP-CDV therapy. |
| | | | | Approximately two weeks after D/C of |
| | | | | HDP-CDV, ADV viremia rebounded. |
| 11 yrs., | EBV- | 4 mg/kg | Con.: GCV | There is no evidence of on-going |
| 27 kg | associated | BIW | | replication of EBV as measured by plasma |
| | PTLD | | | PCR. The patient did have reactivation of |
| | | | | CMV that was treated with GCV. |
| 66 yrs., | ADV | 3 mg/kg first | Prev.: ACV, VALTREX ®, CDV | ADV 35 (urine). 1 to 2 log reductions in |
| 108 kg | nephritis and | dose; | | adenovirus viremia, ADV viruria, CMV |
| (edema) | viremia | 2.5 mg/kg | | viremia, BKV viremia and BKV viruria |
| ~80 kg | BKV | BIW | | were observed. Levels rebounded |
| (normal) | | | | following treatment interruption. |
| 4 yrs., | ADV | 4 mg/kg | Prev.: CDV, ACV, FOS, GCV | ADV2. A ~2 log₁₀ decrease in plasma |
| 14.1 kg | viremia | BIW | Con: ACV, FOS | ADV and a 4 log₁₀ reduction in ADV in |
| | BKV | | | the stool were observed. There was no |
| | viremia | | | obvious decrease in BK or HSV virus |
| | Resistant | | | (HSV resistant to CDV, HDP-CDV, ACV |
| | HSV | | | and FOS) levels. |
| 28 yrs., | CMV | 2 mg/kg | Prev: FOS, GCV, ValGCV | This patient had a modest response in |
| 57.9 kg | viremia | BIW | | CMV viremia but no sustained response of |
| | | | | BKV in urine. |
| 69 yrs., | JCV | 4 mg/kg | Prev.: Mefloquine, CDV | This patient had approximately a 1.5 log₁₀ |
| 55 kg | associated | BIW | | reduction in plasma/serum JCV to below |
| | PML | | | the limit of detection. |
| 66 yrs, | ADV | 3 mg/kg first | Prev.: CDV | After approximately 2-3 weeks of HDP- |
| 108 kg | viremia | dose; | | CDV treatment, ADV viremia decreased |
| | BKV | 2 mg/kg | | by almost 2 log₁₀. BKV in urine rose by a |
| | viremia & | BIW | | similar amount and there was no apparent |
| | viruria | thereafter | | effect on BKV viremia. |
| 48 yrs, | HHV6-A | 4 mg/kg | Prev.: FOS, GCV | This patient received only two doses of |
| 64.4 kg | encephalitis | BIW | | HDP-CDV; there was insufficient time on |
| | | | | treatment to assess virologic response. |
| 2 mo, | Neonatal | 4 mg/kg | Prev.: ACV, FOS, GCV | HSV in the CSF decreased by more than |
| 4.63 kg | HSV | BIW | | 1.8 log₁₀ copies/mL to below the limit of |
| | encephalitis | | | detection. |
| 21 mo, | ADV | 2 mg/kg | Prev.: CDV, ACV | ADV 5 (stool and plasma). Low level |
| 10 kg | viremia | BIW | Con: ACV | baseline plasma viremia (100 copies/mL) |
| | | | | became undetectable and stayed |
| | | | | undetectable until discontinuation of HDP- |
| | | | | CDV when it rebounded to 100 copies/ml. |
| | | | | There was a modest response in stool viral |
| | | | | load (<10-fold). |
| 16 mo, | ADV | 4 mg/kg | Prev.: CDV, CMV IG | ADV 2 (stool and plasma). Plasma ADV |
| 10.4 kg | viremia | BIW | Con: CMV IG | declined from 809 c/ml to undetectable |
| | | | | after 3 weeks of HDP-CDV therapy; stool |
| | | | | ADV declined approximately 1 log₁₀ over |
| | | | | the same time period. |
| 33 yrs, | ADV | 2 mg/kg | Prev.: CDV, VALTREX ® | ADV 5 (plasma). Adenovirus declined 2.6 |
| 65 kg | viremia | BIW | | and 3.6 log₁₀ in plasma and urine |
| | | | | respectively, to the limit of detection. |
| 20 yrs., | ADV | 2 mg/kg | Prev.: ACV, CDV, GCV | ADV 34 (plasma). ADV plasma viremia |
| 67 kg | BKV | BIW | | declined 3.1 logs from 5.1 log₁₀ to the limit |
| | CMV | 4 mg/kg | | of detection; ADV viruria appeared to |
| | | BIW | | respond well initially with a 2.4 log₁₀ |
| | | | | decline only to rebound to within 0.7 log₁₀ |
| | | | | from baseline. Similar trends may be |
| | | | | occurring with BKV and CMV. Notably, |
| | | | | this patient had initially high exposures to |
| | | | | HDP-CDV and then low exposures that |
| | | | | may correlate with these responses. |
| 22 | EBV | 4 mg/kg | Prev.: CDV, GCV | This patient had a steady decline in blood |
| months, | viremia | BIW | | EBV viral load from >50,000,000 |
| 13.8 kg | | | | copies/mL to 100,000 copies/mL over one |
| (dry wt. = | | | | month of HDP-CDV therapy. |
| 10 kg) | | | | |
| 6 months, | EBV | 4 mg/kg | Prev.: GCV | There was approximately a 5-fold decrease |
| 6 kg | associated | BIW | | in EBV levels in blood during the four |
| | PTLD | | | days after starting HDP-CDV in this patient. |
| 55 yrs., | JCV | 4 mg/kg | Prev.: vGCV | No virology data available. |
| 60 kg | associated | BIW | | |
| | PML | | | |
| 50 yrs., | ADV | 2 mg/kg | Prev.: Unknown | ADV 12. Plasma ADV declined 2.4 log₁₀ |
| 81 kg | pneumonia | BIW | | from pre-therapy levels to last day of |
| | | | | dosing (10 days/4doses); there was a |
| | | | | modest decline in stool viral load. |
| 67 yrs., | ADV/EBV | 2 mg/kg | | The only virology data obtained after |
| 75 kg | | BIW | | dosing was within a day of first dose and |
| | | | | therefore inadequate to assess response. |
| 37 yrs., | EBV | 3 mg/kg first | Prev.: vACV | The patient received one dose of HDP-CDV. |
| 72.3 kg | associated | dose | | |
| | with PTLD | followed by | | |
| | | 2 mg/kg | | |
| | | BIW | | |
| 10 yrs., | Disseminated | 4 mg/kg | Prev.: CDV, GCV | ADV 31 (stool). |
| 50 kg | ADV | BIW | | |
| 50 yrs., | Refractory | 2 mg/kg | Prev.: GCV, FOS | The patient received one dose of HDP- |
| 75 kg | CMV | BIW | | CDV. |
| 9 months, | Disseminated | 3 mg/kg | Prev.: CDV | ADV 2 (plasma). ADV in serum increased |
| 6.58 kg | ADV | BIW | | by over a log on therapy while stool |
| | | | | decreased by a similar amount. |
| 39 yrs., | Post Lung | 3 mg/kg | Prev.: GCV, CDV, leflunomide, | There was no change in CMV viremia |
| 72.8 kg | Transplant | BIW | FOS | during therapy with HDP-CDV. Viracor |
| | CMV | | | genotype showing GCV resistance |
| | | | | requested from site. Culture attempts on |
| | | | | virus failed. |
| 4 yrs., | Disseminated | 60 mg BIW | Prev.: CDV | ADV in blood went from 51,000 c/mL to |
| 19.1 kg | ADV | | | 100 c/mL in 4 days after one dose. |
| 11 yrs., | Disseminated | 3 mg/kg | Prev.: CDV | This patient received two doses of HDP- |
| 46 kg | ADV | BIW | | CDV. |
| 4 weeks, | HSV | 4 mg/kg | Prev.: ACV, GCV, FOS | Data pending |
| 4.3 kg | | BIW | | |
| 6 yrs., | ADV | 4 mg/kg | Prev.: CDV | This patient had a 1.2 log₁₀ decrease in |
| Unknown | | BIW | | ADV viremia from 1.5 million to 98,000 |
| | | | | c/ml in the first two weeks of therapy with |
| | | | | 3 doses of HDP-CDV. |
| 62 yrs., | CMV | 3 mg/kg | Prev.: GCV, FOS | GCV res (UL97). There was a 1.9 log₁₀ |
| 61 kg | | BIW | | decrease in CMV viremia over the first |
| | | | | month of dosing with HDP-CDV. |
| 55 yrs., | Refractory | 4 mg/kg | Prev.: FOS, GCV | This patient had evidence of baseline |
| 63.3 kg | CMV | BIW | | resistance to CDV and GCV (UL97 and |
| | | | | UL54 mutations [L501F/L]). CMV |
| | | | | viremia declined from 9200 copies/mL on |
| | | | | first day of dosing to 1000 copies/mL 4 |
| | | | | days after the third dose. Dosing was |
| | | | | interrupted resulting in virologic rebound. |
| | | | | HDP-CDV has been resumed at 4 mg/kg. |
| 8 yrs, | ADV | 100 mg BIW | Prev: CDV | Data pending |
| 24 kg | | | | |

QW = once weekly; BIW = twice weekly; Prev. = Previous; Con. = Concomitant; JCV = JC virus; PML = progressive multifocal leukoencephalopathy; ADV = Adenovirus; ACV = acyclovir; BKV = BK virus; CMV = cytomegalovirus; CDV = cidofovir; FOS = foscarnet; GCV = ganciclovir; EBV = Epstein-Barr virus; PTLD = posttransplant lymphoproliferative disorder; HSV = Herpes simplex virus; HHV = human herpes virus; VIG = vaccinia immune globulin; CSF = cerebrospinal fluid; RBV = ribavirin; ST 246 is 4-trifluoromethyl-N-(3,3a,4,4a,5,5a,6,6a-octahydro-1,3-dioxo-4,6-ethenocycloprop[f]isoindol-2(1H)-yl)-benzamide, see e.g., Antimicrobial Agents and Chemotherapy, May 2008, pp. 1721-1727.

### Example 2

### Tablet Formulations

HDP-CDV free acid tablets (20 mg or 50 mg) were formulated as dry-blend, direct-compressed tablet containing 20 or 50 mg HDP-CDV active ingredient. In addition to the active ingredient, HDP-CDV tablets contained microcrystalline cellulose, mannitol, crospovidone and magnesium stearate. The bioavailability of the tablet formulation was determined in the study described in Example 5.

HDP-CDV tablets of various strengths were developed. The tablets were compressed from a common blend, while varying the drug load for different strengths. The 20 mg, 50 mg and 100 mg dosage forms, respectively, were round, biconvex tablets with dimensions 7.3 mm×3.5 mm, 7.9 mm×3.8 mm, and 10.5 mm×4.4 mm. HDP-CDV as the free acid was formulated as direct compression, instant release tablets containing 20, 50 or 100 mg HDP-CDV (see Tables 3 and 4).

TABLE 3
Composition of 20 mg HDP-CDV Tablets

| | | Amount per Tablet | |
|---|---|---|---|
| Ingredient | Function | % (wt/wt) | mg/tablet |
| HDP-CDV | Active Ingredient | 12.50 | 25.00^{y} |
| Silicified microcrystalline | Diluent, Binder, | 26.88 | 41.50 |
| cellulose | Flow aid | | |
| Mannitol | Diluent | 41.75^{x} | 66.01^{x} |
| Microcrystalline cellulose | Diluent, Binder | 13.38 | 19.41 |
| and Mannitol | | | |
| Crospovidone | Disintegrant | 4.50 | 6.48 |
| Magnesium Stearate | Lubricant | 1.00 | 1.60 |

| | | |
|---|---|---|
| Total: | 100.00 | 160.00 |

^{y}The quantity of HDP-CDV was adjusted based on the drug substance purity factor.
^{x}The target weight of mannitol was adjusted to maintain a constant on a per tablet basis.

TABLE 4
Composition of 50 and 100 mg HDP-CDV Tablets

| | | Amount per Tablet | |
|---|---|---|---|
| Ingredient | Function | %(wt/wt) | mg/g |
| HDP-CDV | Active Ingredient | 27.78 | 277.8^{y} |
| Silicified microcrystalline | Diluent, Binder, | 22.18 | 221.8 |
| cellulose | Flow aid | | |
| Mannitol | Diluent | 34.46^{x} | 344.6^{x} |
| Microcrystalline cellulose | Diluent, Binder | 11.04 | 110.4 |
| and Mannitol | | | |
| Crospovidone | Disintegrant | 3.714 | 37.14 |
| Magnesium Stearate | Lubricant | 0.8253 | 8.253 |

| | | |
|---|---|---|
| Total: | 100.00 | 1000.00 |

^{y}The quantity of HDP-CDV was adjusted based on the drug substance purity factor.
^{x}The target weight of mannitol was adjusted to maintain a constant on a per tablet basis.

### Measurement of Plasma HDP-CDV Concentrations

Plasma concentrations of HDP-CDV and CDV were measured using a validated liquid chromatography mass spectrometry/mass spectrometry (LC-MS/MS) method. The lower limit of quantification (LLOQ) for HDP-CDV in plasma was 0.1 ng/mL and the LLOQ of CDV in plasma was 0.5 ng/mL. The LLOQ may not have been achieved in certain analyses due to insufficient sample volume (<0.5 mL) and the need to dilute the samples prior to analysis.

### Example 3

### Stability Studies

Stability studies for 50 mg and 100 mg tablets were completed using known methods in the art. Tables 5 and 6 show the results for the 50 mg and 100 mg tablets, respectively.

TABLE 5
Stability Data for HDP-CDV Tablets, 50 mg

| | | | 1 Month | |
|---|---|---|---|---|
| Test | Specifications | Initial | 25° C./60% RH | 40° C./75% RH |
| Appearance | White to off-white | White standard bi- | White standard bi- | White standard bi- |
| | standard bi-convex | convex tablets | convex tablets | convex tablets |
| | tablets | | | |
| Identification | Retention time | Retention time | Retention time | Retention time |
| | consistent with | consistent with | consistent with | consistent with |
| | standard | standard | standard | standard |
| Water Content | Report Results | 2.03% | 1.51% | 1.49% |
| Assay | 90.0% to 110.0% of | 99.6% of label claim | 100.6% of label | 102.3% of label |
| | label claim | | claim | claim |
| Related | Report Individual | RRT* 0.64: 0.14% | RRT 0.63: 0.12% | RRT 0.63: 0.12% |
| Substances | Related | RRT 0.83: 0.16% | RRT 0.86: 0.15% | RRT 0.86: 0.15% |
| | Substances: ≧0.05%; | RRT 1.17: <0.05% | RRT 1.33: 0.06% | RRT 1.33: 0.05% |
| | Total Related | RRT 1.33: 0.06% | RRT 2.08: 0.05% | RRT 2.08: 0.05% |
| | Substances: NMT*** | RRT 2.04: 0.05% | Total: 0.38% | Total: 0.37% |
| | 2.5% | RRT 2.09: <0.05% | | |
| | | RRT 2.41: <0.05% | | |
| | | Total: 0.41% | | |
| Dissolution | Report Results at 45 | Avg.: 98% | Avg.: 99% | Avg.: 99% |
| | Minutes | % RSD**: 3.6% | % RSD: 1.9% | % RSD: 1.2% |

*RRT = Rapid Release Tablet
**RSD = Relative Standard Deviation (% RSD)
***NMT = Not More Than

TABLE 6
Stability Data for HDP-CDV Tablets, 100 mg

| | | | 1 Month | |
|---|---|---|---|---|
| Test | Specifications | Initial | 25° C./60% RH | 40° C./75% RH |
| Appearance | White to off-white | White standard bi- | White standard bi- | White standard bi- |
| | standard bi-convex | convex tablets | convex tablets | convex tablets |
| | tablets | | | |
| Identification | Retention time | Retention time | Retention time | Retention time |
| | consistent with | consistent with | consistent with | consistent with |
| | standard | standard | standard | standard |
| Water Content | Report Results | 2.00% | 1.36% | 1.43% |
| Assay | 90.0% to 110.0% of | 102.1% of label | 102.0% of label | 99.4% of label |
| | label claim | claim | claim | claim |
| Related | Report Individual | RRT 0.64: 0.14% | RRT 0.62: 0.12% | RRT 0.61: 0.12% |
| Substances | Related | RRT 0.83: 0.17% | RRT 0.85: 0.15% | RRT 0.85: 0.15% |
| | Substances: ≧0.05%; | RRT 1.17: <0.05% | RRT 1.34: 0.06% | RRT 1.33: 0.05% |
| | Total Related | RRT 1.33: 0.06% | RRT 2.10: <LOQ | RRT 2.10: <LOQ |
| | Substances: NMT | RRT 2.05: 0.05% | Total: 0.33% | Total: 0.32% |
| | 2.5% | RRT 2.09: <0.05% | | |
| | | RRT 2.41: <0.05% | | |
| | | Total: 0.42% | | |
| Dissolution | Report Results at 45 | Avg.: 96% | Avg.: 95% | Avg.: 95% |
| | Minutes | % RSD: 2.7% | % RSD: 0.9% | % RSD: 2.3% |

*RRT = Rapid Release Tablet
**RSD = Relative Standard Deviation (% RSD)
***NMT = Not More Than

### Example 4

### HDP-CDV Monoammonium Salt

HDP-CDV free acid was converted to the monoammonium salt using the following method. A 5 liter round-bottomed flask was equipped with a mechanical stirrer, temperature probe and gas inlet adapter. The flask was charged with HDP-CDV free acid (87.3 g, 0.155 mol), 2-propanol (180 ml) and 28-30% ammonium hydroxide (13 ml). The reaction was stirred and brought to reflux (62-80° C.) to achieve dissolution (10 min). The solution was not allowed to stir for more than 15 min at reflux. The solution was allowed to cool to less than 25° C. for 16±8 h. The mixture was cooled to 5±5° C. for a minimum of 1 h. The product was filtered and washed with chilled 2-propanol (5±5° C., 430 ml). The product, a white solid, was dried at 30-35° C. for 25 h 10 min±2 h. Yield: approximately 86.8 g (0.15 moles) of HDP-CDV monoammonium salt; 96.7% of theoretical based on HDP-CDV free acid.

### Example 5

### Comparative Studies on Bioavailability and Effect of Food

A Phase 1 comparative bioavailability study of HDP-CDV solution versus tablets was carried out. In addition, PK parameters for HDP-CDV and cidofovir (CDV) was compared among subjects who had received HDP-CDV after fasting overnight versus subjects who had received after eating a high fat meal within 30 minutes of dosing. A total of 24 healthy volunteers received three single doses of HDP-CDV in crossover fashion (40 mg solution, fasted; 40 mg tablet following a high fat breakfast; and 40 mg tablet fasted). Each dose was separated by a 14-day washout period. Subjects who received HDP-CDV and had complete concentration-time profiles were included in the PK population for non-compartmental PK analysis, which resulted in a PK population for the study consisting of 24 subjects. All subjects in this study were male. The overall age range for the young volunteers was 19 to 53 years.

### Bioavailability of HDP-CDV Solution Versus Tablets

Following administration of a 40 mg HDP-CDV and CDV solution and tablet (fasted/fed), the mean plasma concentration as a function of time for HDP-CDV and CDV was measured. HDP-CDV was readily absorbed following a single oral administration of both the solution and tablet formulation. While the plasma concentration versus time profile for HDP-CDV was similar for the solution versus the tablet formulation (under fasting conditions), the CDV profile was nearly identical between the two formulations regardless of the presence of food.

HDP-CDV Non-Compartmental PK Parameters: Mean non-compartmental pharmacokinetic parameters for HDP-CDV and CDV following administration of a 40 mg HDP-CDV solution and tablet (fasted) are presented in Table 7 and Table 8, respectively.

TABLE 7
Mean (SD) Plasma Non-Compartmental HDP-CDV Pharmacokinetic Parameters
versus Dose (Solution vs. Tablet Formulation- Fasted)

| HDP-CDV | Cₘₐₓ | Tₘₐₓ | t_{1/2} | AUC₀₋ₗₐₛₜ | AUC_{0-inf} | CL/F | Vz/F |
|---|---|---|---|---|---|---|---|
| Dose | (ng/mL) | (h) | (h) | (h*ng/mL) | (h*ng/mL) | (L/h) | (L) |
| 40 mg | 77.7 | 2.00 | 8.06 | 549 | 574 | 77.5 | 831 |
| solution | (22.9) | (1.00, 4.00) | (3.37) | (159) | (165) | (32.4) | (301) |
| 40 mg | 73.9 | 3.00 | 7.52 | 473 | 496 | 86.1 | 885 |
| tablet | (25.4) | (1.50, 6.02) | (2.74) | (129) | (134) | (22.2) | (238) |

Tₘₐₓ is summarized by median (min, max)

TABLE 8
Mean (SD) Plasma Non-Compartmental Cidofovir Pharmacokinetic Parameters
versus Dose (Solution vs. Tablet Formulation- Fasted)

| HDP-CDV | Cₘₐₓ | Tₘₐₓ | t_{1/2} | AUC₀₋ₗₐₛₜ | AUC_{0-inf} | CL/F | Vz/F |
|---|---|---|---|---|---|---|---|
| Dose | (ng/mL) | (h) | (h) | (h*ng/mL) | (h*ng/mL) | (L/h) | (L) |
| 40 mg | 10.8 | 12.0 | 49.9 | 456 | 505 | 40.8 | 2840 |
| solution | (2.15) | (9.00, 16.0) | (16.5) | (86.4) | (91.0) | (8.16) | (787) |
| 40 mg | 9.85 | 14.0 | 46.9 | 422 | 466 | 44.3 | 2930 |
| tablet | (2.37) | (9.00, 16.0) | (11.5) | (90.1) | (91.9) | (8.95) | (642) |

Tₘₐₓ is summarized by median (min, max)

### Food Effect

Mean non-compartmental pharmacokinetic parameters for HDP-CDV and CDV following administration of a 40 mg HDP-CDV tablet under fasted and fed conditions are presented in Table 9 and Table 10, respectively.

TABLE 9
Mean (SD) Plasma Non-Compartmental HDP-CDV Pharmacokinetic
Parameters versus Dose (Food Effect- Fasted vs. Fed).

| HDP-CDV | Cₘₐₓ | Tₘₐₓ | t_{1/2} | AUC₀₋ₗₐₛₜ | AUC_{0-inf} | CL/F | Vz/F |
|---|---|---|---|---|---|---|---|
| Dose | (ng/mL) | (h) | (h) | (h*ng/mL) | (h*ng/mL) | (L/h) | (L) |
| 40 mg | 73.9 | 3.00 | 7.52 | 473 | 496 | 86.1 | 885 |
| fasted | (25.4) | (1.50, 12.0) | (2.74) | (129) | (134) | (22.2) | (238) |
| 40 mg | 39.4 | 6.00 | 6.65 | 340 | 367 | 119 | 1080 |
| fed | (13.7) | (1.50, 12.00) | (3.01) | (108) | (110) | (41.1) | (447) |

Tₘₐₓ is summarized by median (min, max)

TABLE 10
Mean (SD) Plasma Non-Compartmental Cidofovir Pharmacokinetic
Parameters versus Dose (Food Effect- Fasted vs. Fed)

| HDP-CDV | Cₘₐₓ | Tₘₐₓ | t_{1/2} | AUC₀₋ₗₐₛₜ | AUC_{0-inf} | CL/F | Vz/F |
|---|---|---|---|---|---|---|---|
| Dose | (ng/mL) | (h) | (h) | (h*ng/mL) | (h*ng/mL) | (L/h) | (L) |
| 40 mg | 9.85 | 14.0 | 46.9 | 422 | 466 | 44.3 | 2930 |
| fasted | (2.37) | (9.00, 16.0) | (11.5) | (90.1) | (91.9) | (8.95) | (642) |
| 40 mg | 9.24 | 14.0 | 45.5 | 407 | 452 | 46.9 | 2980 |
| fed | (2.18) | (9.00, 16.0) | (14.4) | (105) | (109) | (12.9) | (841) |

Tₘₐₓ is summarized by median (min, max)

The results indicate that food affects the absorption and bioavailability of HDP-CDV, as evidenced by a reduced peak concentration and delayed Tₘₐₓ in the presence of a high fat meal. Food also affected the overall exposure of HDP-CDV in plasma as shown by reduced mean AUC values in the fed state. In contrast, CDV was unaffected by the presence of food.

FDA-guidance specified two-one sided tests (TOST) were performed to confirm these findings. For HDP-CDV, the results presented in Table 11 indicate that all of the confidence intervals are outside of the standard equivalence interval with even the upper 90% CI falling outside of the 0.8 to 1.25 range. These results suggest that there is a significant food effect on the HDP-CDV tablet formulation and serious consideration should be given to instruct patients to only take the drug on an empty stomach. Results from the CDV food-effect arm presented in Table 12 indicate that all of the confidence intervals are inside the standard equivalence interval with all of the 90% CI including 100%. These results suggest that there is no food effect on CDV concentrations when CDV is administered in the tablet formulation.

TABLE 11
HDP-CDV Fed Tablet vs. Fasted Tablet Two-One Sided Tests

| | | Ref | Test | | | | |
|---|---|---|---|---|---|---|---|
| | | Geometric | Geometric | Ratio | 90% CI | 90% CI | |
| Parameter | Units | LSM | LSM | [% Ref] | Lower | Upper | Power |
| Ln(AUC_{inf}) | h*ng/mL | 475.9849 | 343.5310 | 72.17 | 65.38 | 79.67 | 0.9798 |
| Ln(AUCₗₐₛₜ) | h*ng/mL | 456.8364 | 323.8453 | 70.89 | 64.00 | 78.51 | 0.9736 |
| Ln(Cₘₐₓ) | ng/mL | 70.4679 | 37.0360 | 52.56 | 46.39 | 59.54 | 0.9041 |

TABLE 12
Cidofovir Fed Tablet vs. Fasted Tablet Two-One Sided Tests

| | | Ref | Test | | | | |
|---|---|---|---|---|---|---|---|
| | | Geometric | Geometric | Ratio | 90% CI | 90% CI | |
| Parameter | Units | LSM | LSM | [% Ref] | Lower | Upper | Power |
| Ln(AUC_{inf}) | h*ng/mL | 455.9056 | 437.5598 | 95.98 | 90.49 | 101.80 | 1.0000 |
| Ln(AUCₗₐₛₜ) | h*ng/mL | 412.5662 | 393.2650 | 95.32 | 89.61 | 101.40 | 1.0000 |
| Ln(Cₘₐₓ) | ng/mL | 9.5796 | 8.9710 | 93.65 | 87.59 | 100.12 | 0.9999 |

### Example 6

### Pharmacokinetic Analysis in Pediatric Patients

This study population included five children aged 4, 11, 12, 14, and 16 years. Pediatric patients received approximate doses of 1, 2, 3, or 4 mg/kg given once or twice weekly. These patients had a wide range of underlying medical conditions. Variability in PK parameters for this age group was high with respect to AUC, Cₘₐₓ, and CL/F. In general, pediatric patients had lower plasma concentrations of HDP-CDV and CDV than adult healthy volunteers or patients on the same dose and schedule.

The study population also included 3 elderly adults aged 66, 67, and 68 years. Geriatric patients received approximate doses of 1, 2, or 3 mg/kg given either once or twice weekly. Similar to pediatric patients, the medical condition of geriatric patients varied widely. Variability in PK parameters for this age group was high with respect to AUC, Cₘₐₓ, and CL/F. In general, geriatric patients had higher plasma concentrations of HDP-CDV and CDV and lower CL/F than those observed in younger healthy volunteers or patients.

The study patients <20 years of age tended to have higher CL/F values than older subjects, with 2 of the 3 oldest patients having lower CL/F values. High CDV CL/fm values were also observed in children.

### Example 7

### Monitoring Development of AdV Disease, BKV Infection, and EBV Associated Syndromes

A multicenter, randomized, double-blind, placebo-controlled, dose-escalation study of safety, tolerability, and ability of HDP-CDV to prevent or control AdV, BKV, and EBV infection and associated disorders was conducted. During enrollment of patients in the study, urine samples of candidate patients were routinely screened for BKV. Plasma was collected from patients who tested positive for BKV viruria in urine, and AdV, EBV and BKV level in the plasma was evaluated.

### Study Cohorts

Enrolled study population was divided into five cohorts:

Cohort 1: 40 mg HDP-CDV versus placebo QW; 40 subjects randomized.

Cohort 2: 100 mg HDP-CDV versus placebo QW; 39 subjects randomized.

Cohort 3: 200 mg HDP-CDV versus placebo QW; 53 subjects randomized.

Cohort 4: 200 mg HDP-CDV versus placebo BIW initially, then reduced to 200 (QW); 40 subjects randomized.

Cohort 4A: 100 mg HDP-CDV versus placebo BIW; 67 subjects randomized.

Baseline Subject Characteristics for the end-organ damage and/or treatment associated with the treatment of BKV infection is described in Table 13.

TABLE 13
Baseline Subject Characteristics of the study population

| | BK positive during treatment | | BK negative throughout | |
|---|---|---|---|---|
| | HDP-CDV | Placebo | HDP-CDV | Placebo |
| | N = 94 | N = 32 | N = 77 | N = 27 |
| Age (years) | | | | |
| Mean | 49.9 | 49.9 | 51.8 | 50.7 |
| Median | 50.0 | 50.0 | 55.0 | 53.0 |
| Min, Max | 23, 71 | 21, 68 | 22, 70 | 26, 70 |
| Gender, n(%) | | | | |
| Female | 37 (39.4) | 12 (37.5) | 36 (46.8) | 13 (48.2) |
| Male | 57 (60.6) | 20 (62.5) | 41 (53.3) | 14 (51.9) |
| Race, n(%) | | | | |
| Asian | 6 (6.4) | 1 (3.1) | 4 (5.2) | 1 (3.7) |
| Black | 1 (1.1) | 4 (12.5) | 2 (2.6) | 0 |
| White | 84 (89.4) | 27 (84.4) | 70 (90.9) | 26 (96.3) |
| Other | 3 (3.2) | 0 | 1 (1.3) | 0 |
| Weight (kg) | | | | |
| Mean | 77.7 | 84.7 | 77.1 | 71.2 |
| Median | 77.7 | 83.3 | 77.6 | 70.8 |
| Min, Max | 40.6, 131.9 | 45.4, 146.9 | 45.5, 127.5 | 40.8, 110.7 |
| Unrelated donor, n(%) | 51 (54.3) | 16 (50.0) | 46 (59.7) | 11 (40.7) |
| Adult mismatch, n(%) | 15 (16.0) | 1 (3.1) | 12 (15.6) | 5 (18.5) |
| Myeloablative | 61 (64.9) | 22 (68.8) | 48 (62.3) | 16 (59.3) |
| Conditioning, n(%) | | | | |
| Pre-dose | 44 (46.8) | 19 (59.4) | 26 (33.8) | 7 (25.9) |
| Cyclophosphamide, n(%) | | | | |
| Pre-dose Steroids, n(%) | 42 (44.7) | 16 (50.0) | 27 (35.1) | 11 (40.7) |

### Measurement of BK Viruria Prior to and During Treatment

All but one subject had BK viruria prior to dosing in the study population (cohorts 1-4A). During the study period, BKV in the urine was in the range between 4.7×10⁹ to 2.0×10¹⁰ copies/mL, and, among this population, all but one had BK viremia (median 1.3×10³; range 500 to 1.7×10⁵ copies/mL).

HDP-CDV treatment was associated with a decreased frequency of BKV associated adverse bladder events. 17 subjects (7.4% of the total subject population) were confirmed as developing blood positive on routine urinalysis during the treatment period. 12/17 (70.6%) had BK viruria, with 11 documented at pre-dose; 1 developing BK viruria during the course of treatment.

There was a higher rate of blood positive urinalyses occurring during the treatment period in placebo-treated subjects (15.2%) compared to those treated with HDP-CDV (4.7%). There was nearly a 6-fold difference between the pooled HDP-CDV- and pooled placebo-treated groups, when the rates of confirmed blood positive urinalyses were compared for subjects with BKV viruria during the treatment period: 8/59 (13.6%) placebo-recipients and 4/171 (2.3%) for HDP-CDV-treated subjects, respectively. In contrast, the rates of blood positive urinalyses in subjects without BK viruria were low and comparable (1.7% versus 2.4% for placebo- and HDP-CDV-treated subjects, respectively).

The study showed that HDP-CDV treatment was associated with a reduction in microscopic hematuria in subjects shedding BKV in their urine. Only 14% (10/73) of HDP-CDV treated subjects were confirmed to have developed new onset BK viruria in comparison to 25% (7/28) of placebo-treated subjects during treatment; moreover, when sustained BK viruria is considered (defined as at least 4 weeks with BK positive urine measurements), 21% of placebo-treated subjects were affected, compared to 12% of HDP-CDV-treated subjects. In summary, HDP-CDV treatment decreased the incidence of BKV-related bladder events.

About 54% (125/230) of the subjects had measureable BKV in urine samples at some time during the treatment period. Of these, 81% (101/125) were BKV positive at baseline. The 101 subjects entering the study with BKV positive urine measurements had sustained BK viruria on subsequent visits. In the HDP-CDV cohorts, 77/171 (45%) of subjects were positive at baseline as compared to 24/59 (41%) of those in the placebo group. Nearly all subjects with BKV positive urine at baseline entered the study with BK viruria of >10,000 copies/mL (72/77 [93%] and 23/24 [96%] of HDP-CDV- and placebo-treated subjects respectively) and all had sustained viruria (defined as at least 5 positive urine measurements during the treatment period [through post-dose Week 1]).

Subjects with high urine values of BKV (>1.0E+10 copies/mL; >1×10¹⁰) were identified in all cohorts and the pooled placebo group. There was an imbalance in subjects entering the study with BK viruria, with 23/39 (60%) belonging to cohort 3, as compared to percentages ranging from 32% to 44% across the other cohorts and the pooled placebo group. When viruria >1×10¹⁰ copies/mL at baseline was examined, 8/13 (61%) subjects had been randomized to Cohort 3. However, when the proportions of subjects who developed high levels of viruria (defined as >1×10¹⁰ copies/ml) during the treatment period were assessed, the incidence ranged from 9% to 25%, without clear relationship to dose or treatment assignment.

BK viremia also occurred in subjects across all groups, ranging from 23% (5/22) in Cohort 4a to 62% (5/8) in cohort 1. Very few subjects developed viremia >10,000 copies/mL (1, 1, 3, 3, 0 and 0 in cohorts 1 through 4a and placebo, respectively).

Therefore, there was a high incidence of BK viruria in subjects at the time of enrollment, which persisted during the active treatment period.

### Hematuria and Hemorrhagic Cystitis

BK viruria has been associated with hematuria and hemorrhagic cystitis, although the frequency of clinically significant events is rare (O'Donnell 2009; de Puada Silva 2009; Giraud 2006). There were 9 subjects with adverse events (AEs) related to clinically significant BKV bladder events (7 subjects with BK hemorrhagic cystitis and 2 subjects reported with blood in urine in association with BKV). All but one subject had BK viruria prior to dosing into Study HDP-CDV-201. During the treatment period, all patients had high BK urine measurements (median 2×10¹⁰; range 4.7×10⁹ to 2.0×10¹⁰) and all but one had BK viremia (median 1.3×10³; range 500 to 1.7×10⁵). Of these subjects, 3/59 (5.1%) were placebo-treated. Therefore, although the overall incidence was small, HDP-CDV treatment appeared to be associated with a decreased frequency of BKV bladder events reports as adverse events (AEs).

There were 17 subjects (7.4% of the total population of 230 subjects) confirmed as developing 1+ blood on urinalysis during the treatment period. The majority of these subjects (12/17 [70.6%]) had BK viruria, with 11 documented at pre-dose and 1 developing BK viruria during the course of treatment. There was a higher rate of blood positive urinalyses in placebo-treated subjects (9/59 [15.2%]) compared to those treated with HDP-CDV (8/171 [4.7%]).

There was a nearly a 6-fold difference between the pooled HDP-CDV- and placebo-treated groups when the rates of confirmed blood positive urinalyses were compared for subjects who had BKV viruria during the treatment period: 8/59 (13.6%) placebo-recipients and 4/171 (2.3%) for HDP-CDV treated subjects, respectively. In contrast, the rates of blood+urinalyses in subjects without BK viruria were low and comparable (1.7% versus 2.4% for placebo- and HDP-CDV-treated subjects, respectively). These observations suggest that HDP-CDV treatment may be associated with a reduction in microscopic hematuria in subjects shedding BKV in their urine. With respect to subjects who enrolled in the study with no BK viruria at baseline, there were very few instances of high level BK viruria (>1×10¹⁰ copies/mL) or viremia reported, although detectable BK viruria was documented in both HDP-CDV- and placebo-treated subjects during the course of study. When the duration of viruria was assessed, there appeared to be a reduction in the incidence of sustained viruria when comparing the pooled HDP-CDV- and placebo-treated groups, especially with the BIW dosing regimens.

To further explore the potential benefit of HDP-CDV in preventing the development of high-level BKV viruria, data were tabulated for subjects initially negative for BKV in urine samples at baseline.

Subjects who were initially negative for BK viruria ranged from 41% to 76% across the groups. Of those who had at least 4 measurements reported (one of which was from the posttreatment Week 1 visit), 16/73 (22%) and 8/28 (29%) of subjects developed measurable BK viruria on at least one occasion in the pooled HDP-CDV- and placebo-treated groups, respectively. Of note, only 14% (10/73) of HDP-CDV treated subjects were confirmed to have BK viruria in comparison to 25% (7/28) of placebo-treated patients; moreover, when sustained BK viruria is considered (defined as at least 4 weeks with BK positive urine measurements), 21% (6/28) of placebo-treated subjects were affected, compared to 12% (9/73) of HDP-CDV-treated subjects. Very few subjects had high levels of BK viruria (0, 1, 2, 0, 0 and 1 subject in Cohorts 1 through 4a and placebo, respectively) and only one subject each had viremia in the HDP-CDV- and placebo-treated groups.

The following observations were made: 125/230 (54%) of subjects had measurable BK viruria at some time during the active treatment period. Approximately 44% (101/230) of these (HDP-CDV and placebo combined) had BK viruria upon entry, with the majority having values of 10,000 copies/mL or greater. Approximately 27% (27/101) had at least one extremely high measurement of BK viruria (>1×10¹⁰ copies/mL) during the treatment period. 13% (13/101) of these subjects had these very high levels of BK viruria upon entry and an additional 14% developed similar levels during the treatment period. 3/59 (5.1%) of placebo-treated subjects were reported to have BK-related bladder events (cystitis or blood in urine), compared to 6/171 (3.5%) who had received HDP-CDV. For the latter subjects with bladder AEs, only 4/171 (2.3%) had received HDP-CDV doses associated with antiviral activity against another ds DNA virus (CMV) (i.e., doses >100 mg QW); the remaining 2 received 40 mg QW, which has not been shown to have antiviral activity. All but one of the subjects with BK-related bladder events (cystitis or blood in urine) were from the subgroup of subjects who had BK viruria >1×10¹⁰ copies/mL at entry into Study HDP-CDV-201. With respect to laboratory reports of blood in urine, 7.4% (17/171) of subjects developed confirmed ≧1+ blood in urinalyses during the treatment period. 71% of these subjects had BK viruria during the treatment period.

HDP-CDV-treatment had a beneficial effect on BK associated bladder events. First, very high BK viruria measurements (≧1×10¹⁰ copies/mL) were associated with the subjects with clinically important events (AEs for cystitis or blood in urine); Second, the rates of confirmed blood positive urinalyses occurred at ⅙^{th} the rate in HDP-CDV-compared to placebo-treated subjects; and third the incidence of sustained BK viruria was reduced for HDP-CDV subjects who developed BK viruria during treatment.

### Serum Creatinine and Heme as Markers of Renal Function

Serum creatinine concentrations as a marker of renal function were evaluated in this study. Creatinine level more than 120 microM (1.36 mg/ml) was considered elevated. Percent change from baseline in creatinine level was calculated and a 25% increase was considered clinically important change during the treatment period.

Microscopic hematuria was evaluated using heme +1 urinalysis as a surrogate. End of treatment (last value) elevations in serum creatinine measurements (>120 microM (1.36 mg/dl)) were considered clinically meaningful. In order to separate out potentially pre-existing renal dysfunction, both a last measured value for creatinine of >120 microM and at least a 25% increase from baseline was defined as a renal event of potential interest.

In the 125 subjects who were BK viruric during the treatment period, there was a beneficial effect of HDP-CDV treatment in reducing the incidence of renal dysfunction (creatinine elevations) by 2.5 fold. 7/93 (7.5%) of HDP-CDV-treated and 6/32 (18.75%) of placebo-treated subjects had end of treatment creatinine elevations >120 microM and =>25% over baseline values. In addition, in these BK-positive subjects, there was a 3.2 fold difference in frequency between treatments when subjects with creatinine elevations (as defined above) or new onset heme+urinalyses were tallied: 11/93 (11.8%) of HDP-CDV-treated and 12/32 (37.5%) of placebo-treated subjects had either event during the treatment period. In contrast, in the 105 subjects who were consistently BK negative throughout the treatment period, the rates for either creatinine elevations or the combined analysis of creatinine or heme+urine were numerically similar. This lack of effect in BK-negative subjects suggests a specific action of HDP-CDV treatment on BK-associated events.

Baseline data and initial creatinine levels 125 subjects were BK viruria positive at some point during treatment; 105 subjects were consistently BK negative. 26/126 (20.8%) of BK-positive subjects had a Creatinine >120 microM at baseline, compared to 10/105 BK-negative subjects (9.5%). Initial creatinine elevations tended to resolve over time in both groups. Only 1 of the initially elevated subjects had a 25% increase during the treatment period. Last value (post week 1 or last available) 25/125 (20.0%) of BK-positive subjects and 13/105 (12.3%) of BK-negative subjects had elevated creatinines at last observation. Of these, 13/125 (10.4%) of BK-positive and 9/105 (8.6%) of BK negative subjects had creatinine elevations of >120 microM and =>25% increase from baseline. All but one of these subjects developed the initial elevation after enrollment in the study. While there appeared to be similar frequencies of creatinine elevations between BK-positive and BK-negative groups as a whole, the distributions within the BK positive group with respect to HDP-CDV- and placebo-treatment effects appeared to be quite different. In the BK-negative group, rates were similar: 7/78 (9.0%) of HDP-CDV- and 2/27 (7.4%) of placebo-treated subjects had elevations >120 microM and =>25% increase over baseline. In contrast, in the BK-positive group, there was a 2.5-fold increase in incidence in the placebo group: 7/93 (7.5%) of HDP-CDV- and 6/32 (18.75%) of placebo-treated subjects had clinically important creatinine elevations (>120 microM and =>25% over baseline). BK virus can have effects on renal function and the bladder (hematuria, cystitis, dysuria etc.). While not systematically evaluated in Study HDP-CDV-201, an analysis of routine laboratory values (serum creatinine elevations and the presence of new onset, confirmed hematuria) were considered as potential markers of BK effects in these subjects post HSCT. Very few subjects had both creatinine elevations (>120 microM at last value) and confirmed new onset 1+ heme on urinalysis: 4/125 BK-positive and 1/105 BK negative subjects. When subjects were tallied by having either creatinine elevations as defined OR confirmed heme+urinalyses, the frequencies for each pooled treatment group were similar for BK negative subjects [11/78 (14.1%) of HDP-CDV and 3/27 (11.1%) of placebo-treated subjects].

In comparison, there was a 3.2 fold difference in frequency between treatments when BK positive subjects were considered: 11/93 (11.8%) of HDP-CDV- and 12/32 (37.5%) of placebo-treated subjects had either creatinine elevations (=>120 microM at last value &=>25% increase over baseline) OR confirmed, new onset heme +1 urinalysis results during the treatment period.

These observations and other related tallies are included in Table 14.

TABLE 14

| | BK POSITIVE | | | | | |
|---|---|---|---|---|---|---|
| | DURING | | | BK NEGATIVE | | |
| | TREATMENT | | | THROUGHOUT | | |
| | N = 125 | | BK | N = 105 | | BK |
| | DRUG | PLACEBO | POSITIVE | DRUG | PLACEBO | NEGATIVE |
| | 93 | 32 | 125 | 78 | 27 | 105 |
| CR >120 BASELINE* | 16/93 | 10/32 | 26/125 | 9/78 | 1/27 | 10/105 |
| | 20.4% | 31.2% | 20.8% | 11.5% | 3.7% | 9.5% |
| # THAT INCREASED | 0 | 0 | 0 | 1 | 1 | 2/105 |
| DURING TREATMENT | | | | | | 2.3% |
| # THAT INCREASED >25% | 0 | 0 | 0 | 1 | 0 | 1/105 |
| CR >120 LAST VALUE | 14/93 | 11/32 | 25/125 | 10/78 | 3/27 | 13/105 |
| | 15.1% | 37.5% | 20.0% | 12.8% | 11.1% | 12.3% |
| >25% INCREASE FROM | 7/93 | 6/32 | 13/125 | 7/78 | 2/27 | 9/105 |
| BASELINE & >120 AT | 7.5% | 18.75% | 10.4% | 9.0% | 7.4% | 8.6% |
| LAST VALUE** | | | | | | |
| CREATININE >120 | 10/93 | 7/32 | 17/125 | 6/78 | 2/27 | 8/105 |
| LAST VALUE AND NEW | 10.8% | 21.9% | 13.6% | 7.7% | 7.4% | 7.6% |
| FROM BASELINE*** | | | | | | |
| >120; NEW; >25% | 7/93 | 6/32 | 13/125 | 6/78 | 2/27 | 8/105 |
| INCREASE**** | 7.5% | 18.75% | 10.4% | 7.7% | 7.4% | 7.6% |
| 1 + HEME CONFIRMED; | 4/93 | 8/32 | 12/125 | 4/78 | 1 | 5/105 |
| NEW ONSET | 4.3% | 25% | 9.6% | 5.1% | 3.7% | 4.8% |
| CR >120 LAST VALUE; | 1/93 | 3/32 | 4/125 | 1/78 | 0 | 1/105 |
| 1 + HEME CONFIRMED | 1.1% | 9.4% | 3.2% | 1.3% | | 1.0% |
| NEW ONSET | | | | | | |
| >120 LAST; 1 + HEME | 1/93 | 2/32 | 3/125 | 1 | 0 | 1/105 |
| NEW ONSET; | 1.1% | 6.35% | 2.4% | 1.3% | | 1.0% |
| CONFIRMED; >25% INC | | | | | | |
| CREATININE | | | | | | |
| >120 LAST AND >25% | 11/93 | 12/32 | 23/125 | 11/78 | 3/27 | 14/105 |
| INCREASE FROM | 11.8% | 37.5% | 18.4% | 14.1% | 11.1% | 13.3% |
| BASELINE OR 1 + HEME, | | | | | | |
| CONFIRMED, NEW ONSET | | | | | | |

*majority ended up resolving during treatment period
**all that increased during treatment period to be >120 microM
***new onset elevations
****new onset, >25% of baseline value

### Example 8

### Retrospective Study of BKV Associated End-Organ Damage

The subjects in the cohort were retrospectively analyzed for incidence of BK virus infection and effect on end-organ complications. Subjects enrolled in the study had BK viruria measured at every visit and viremia assessed if viruria was present. Data from the study were retrospectively analyzed to assess whether HDP-CDV had an effect of BKV infection end-organ diseases. Microscopic hematuria was defined as confirmed heme positive urinalyses; renal impairment was defined as having an elevated creatinine (≧120 μmol/L) on the last measurement during treatment that was also ≧25% increased from Baseline.

In the clinical study, 230 subjects were enrolled in the study; 59 received placebo and 171 received HDP-CDV, at various doses. 24 subjects (41%) on placebo and 77 subjects on HDP-CDV (45%) had BK viruria prior to dosing. Of the 77 HDP-CDV subjects who were BK viruria positive (BKU+) at Baseline, 4 (5%) developed Hem+ during the Study, versus 8/24 (33%) of the BKU+ placebo recipients. By contrast, in BKU− subjects, 4% of the HDP-CDV- and placebo-recipients were each Hem+. In BKU+ subjects, 8% of HDP-CDV-recipients and 13% of placebo-recipients developed clinical bladder adverse events related to BKV. In BKU+HDP-CDV subjects 10/77 (13%) developed renal impairment versus 7/24 (29%) placebo-recipients. By contrast, in BKU− subjects renal impairment was rare and observed in 6/94 (6%) and 2/35 (6%), of HDP-CDV and placebo-recipients, respectively. Twice weekly dosing was equally or more effective.

### DEFINITIONS AND METHODS

Clinically meaningful end organ effects were defined as follows:
Microscopic hematuria—at least 1+ heme noted on urinalysis (dipstick)
New onset hematuria—at least 1+ heme (confirmed by a consecutive measure of ≧Trace), occurring during treatment only
Renal dysfunction at end of treatment—serum creatinine ≧120 μM (≧1.36 mg/dl) at the end of treatment
New onset renal dysfunction—serum creatinine ≧120 μM (≧1.36 mg/dl) at the end of treatment AND at least 25% greater than baseline serum creatinine

Subjects were tabulated according to treatment group (pooled HDP-CDV versus placebo) and BKV status (viruria positive or negative any time during treatment). Pairwise comparisons were performed using a Fisher's exact test. Data were pooled for HDP-CDV versus placebo groups due to the limited sample size. This approach is conservative wince the 40 mg QW dose had no antiviral effect. Data from the largest study cohort (HDP-CDV 100 mg BIW) are presented separately in some of the analyses as this dose as been selected for further development of HDP-CDV in Phase 3 trials.

Among 230 patients, 126 (54.8%) had measureable BKV in urine samples at some time during treatment. Of these, 101/126 (80%) were positive at baseline. Cyclophosphamide use as part of the conditioning regimen and male gender were the only parameters differing between subjects with and without BKV infection during the study, consistent with previous reports.

Table 15 presents the extent of BKV viral replication in subjects randomized to HDP-CDV (including 100 mg BIW) and placebo.

TABLE 15
Extent of Viruria and Viremia in Subjects
having positive BKV viruria

| | Cohort 4A | Pooled | |
|---|---|---|---|
| | HDP-CDV | HDP- | |
| | 100 mg | CDV | Pooled |
| | BIW | cohorts | Placebo |
| | N = 50 | N = 171 | N = 59 |
| Positive urine at | 22/50 | 77/171 | 24/59 |
| baseline | (44%) | (45%) | (41%) |
| Viruria >1.0E+7 at | 15/22 | 52 | 15 |
| baseline | (68.2%) | (67.5%) | (62.5%) |
| Viruria >1.0E+10 at | 1/22 | 11/77 | 4/24 |
| baseline | (4.5%) | (14.3%) | (16.7%) |
| Viruria >1.0E+10 at | 4/22 | 26/77 | 8/24 |
| any time during | (18.2%) | (33.8%) | (33.3%) |
| treatment | | | |
| Viruria >1.0E+10 but | 1/22 | 4/77 | 1/24 |
| only at posttreatment | (4.5%) | (5.2%) | (4.2%) |
| week | | | |
| Viremia at baseline | 2/22 | 11/77 | 3/24 |
| | (9.0%) | (14.3%) | (12.5%) |
| Viremia at any time | 6/22 | 40/77 | 15/24 |
| during treatment | (27.3%) | (47%) | (46%) |
| Viremia sustained (at | 5/22 | 32/77 | 7/24 |
| least 2 consecutive | (22.7%) | (41.6%) | (29.2%) |
| values positive) | | | |
| Viremia sustained, but | 3/22 | 23/77 | 4/24 |
| no viremia at baseline | (13.6%) | (29.9%) | (16.7%) |
| Viremia >1000 | 4/22 | 31/77 | 9/24 |
| copies/mL confirmed | (18.2%) | (26%) | (37.5%) |
| >10,000 confirmed, | 0 | 5/77 | 0/24 |
| new onset | | (6.5%) | |

To explore the impact of BKV infection on emergence of symptoms, the incidence of urinary AEs containing the term BKV in subjects with BKV infection prior to dosing (i.e., BKV PCR positive at Baseline) was first analyzed, such AEs were rare and reported in 6/77 (7.8%) in subjects who received HDP-CDV as compared to 3/24 (12.5%) of subjects who received placebo.

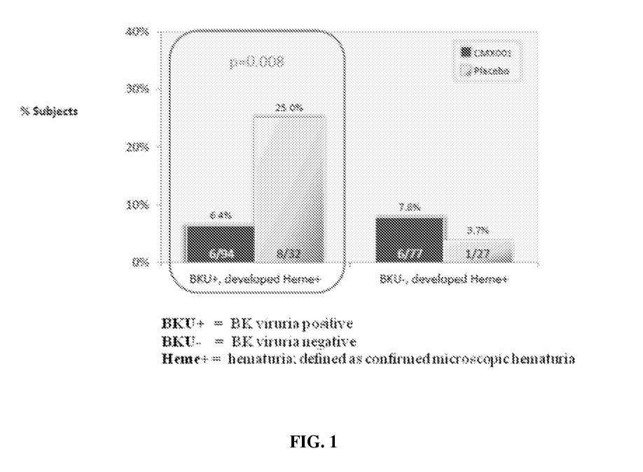

To further explore the impact of HDP-CDV on hemorragic cystitis emergence, the incidence of treatment emergent hematuria was explored. The data in FIG. 1 shows that the HDP-CDV prevented Hematuria (Hem+) in HSCT patients with BKV at base line (BKU+), compared to the placebo group. There was no significant difference in Hem+ in patients who are BKV viruria negative (BKU−) at baseline compared to the placebo group. See FIG. 1.

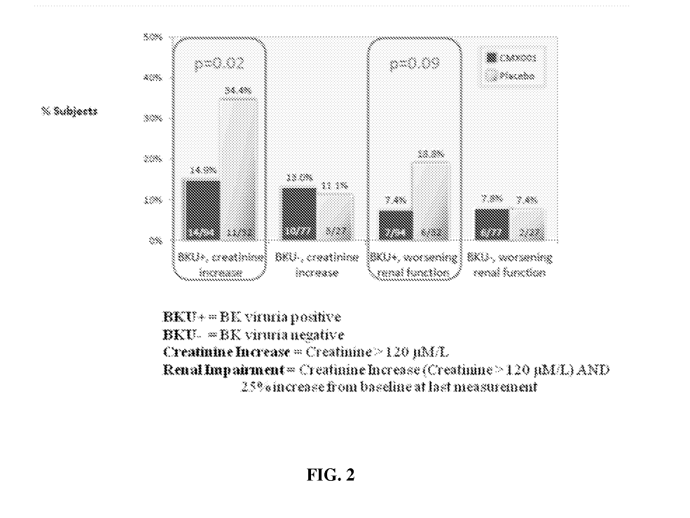

The impact of HDP-CDV on renal dysfunction in subjects with preexisting BKV infection is presented in FIG. 2. The data shows that HDP-CDV prevented increase in Creatinine level and worsening of renal function in patients who were BKV viruria (BKU+) at baseline (post-HSCT engraft) compared to the placebo group. HDP-CDV did not impact the end organ damage in patients who were BKV viruria negative (BKU−) at baseline. In these patients, the Creatinine level did not increase compared to the placebo group. See FIG. 2.

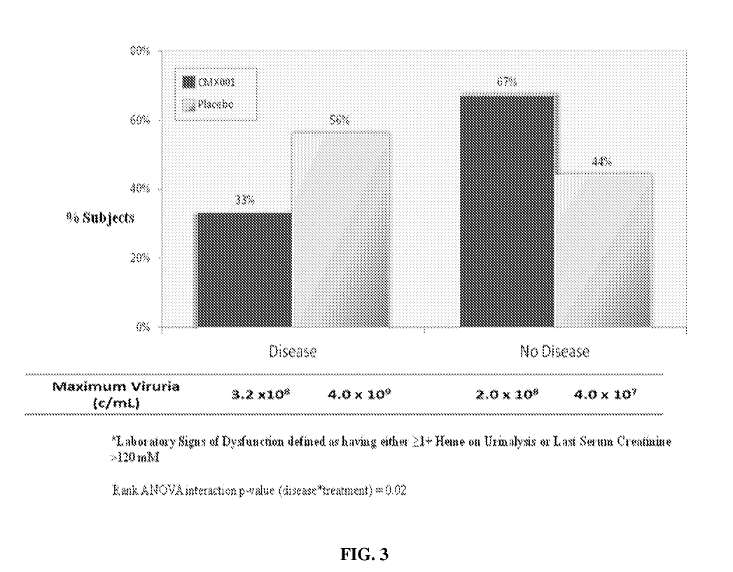

The relationship between maximum BKV viruria and end organ damage by treatment group is shown in FIG. 3. HDP-CDV was effective in reducing BKV viral load and the risk or incidence of end organ damage in BKU+ patients. Among patients who developed end organ disease after treatment (33%), the viral load was significantly higher compared to patients (67%) who did not develop end organ disease (3.2×10⁸ vs. 2.0×10⁸). See FIG. 3. Moreover, patients who developed end organ disease after treatment with HDP-CDV had significantly lower viral load compared to patients who received placebo. See FIG. 3.

### Example 9

### Prevention of Varicella-Zoster Virus spread by HDP-CDV

The alpha-herpesvirus, varicella-zoster virus (VZV), was extremely sensitive to HDP-CDV in culture (EC₅₀=0.4 nM) and was highly effective in vivo using the SCID-Hu mouse model of VZV replication. The VZV-BAC-Luc strain was grown in human skin xenografts and expression level was measured by bioluminescence imaging. Four mouse studies (N=157 total) were performed to evaluate the optimal HDP-CDV dose and treatment duration. HDP-CDV was suspended in 0.4% carboxymethylcellulose (vehicle) and 8 doses were given by oral gavage once daily starting 2 dpi. Doses of 1.25, 2.5, and 5.0 mg/kg were effective and significantly prevented VZV spread (1-way ANOVA, p<0.0001); viral load was dose dependent (qPCR). In contrast, acyclovir (120 mg/kg p.o. BID) was not effective in vivo in this study. Eight doses of 5 mg/kg HDP-CDV delayed to 2 dpi or 4 dpi, and 4 doses from 2-5 dpi, were effective. Indeed, a single dose of 20 mg/kg given on Day −1, 2, or 4 prevented VZV spread for 5 days. Virus rebounded, although spread was reduced 10-fold. Similarly, 1 day of HDP-CDV (20 μg/mL) in the medium of VZV-infected cultures reduced virus spread by 2-3 Log₁₀ when given either on Day −1, 0, 2, or 3. The long intracellular half-life of the active antiviral anabolite of HDP-CDV provides long-lasting antiviral activity, which greatly increases the therapeutic potential for serious zoster infections. HDP-CDV may be used to treat difficult zoster infections and prevent post-herpetic neuralgia.

### EQUIVALENTS

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method of delaying onset, reducing risk, or treating end-organ damage or impairment in a subject infected with BK virus, the method comprising orally administering to the subject a pharmaceutical composition comprising a therapeutically effective dose of a compound selected from: 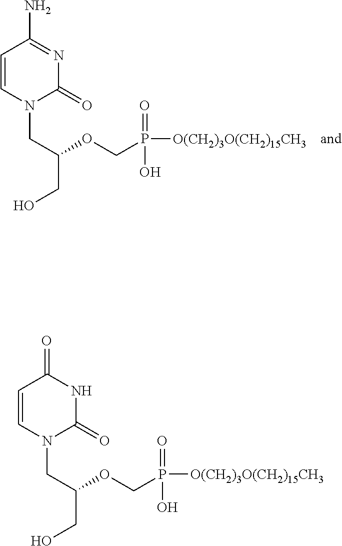
or a pharmaceutically acceptable salt thereof.

2. The method according to claim 1, wherein said subject is a post-hematopoietic stem cell transplant (HSCT) subject.

3. The method according to claim 1, wherein said end organ is selected from kidney, ureter, urinary bladder, prostate, and urethra.

4. A method of reducing incidence of hematuria or renal impairment in a subject at risk of BK virus infection reactivation, the method comprising orally administering to the subject a pharmaceutical composition comprising a therapeutically effective dose of a compound selected from: 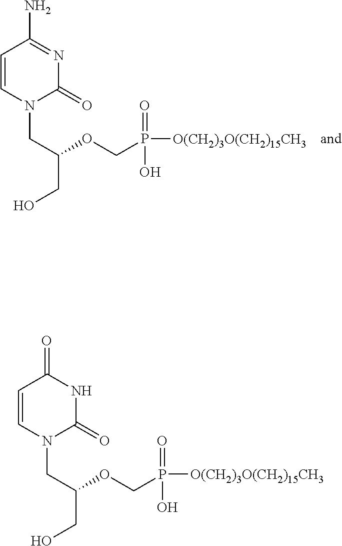
or a pharmaceutically acceptable salt thereof.

5. The method according to claim 4, wherein said subject is a post-hematopoietic stem cell transplant (HSCT) subject.

6. The method according to claim 5, wherein said pharmaceutical composition further reduces BK virus infection reactivation in said subject.

7. The method according to claim 6, wherein said pharmaceutical composition lowers BK viral load in said subject.

8. The method according to claim 7, wherein said pharmaceutical composition delays onset of or reduces risk of end-organ damage or impairment, wherein the end organ is selected from kidney, ureter, urinary bladder, prostate, and urethra.

9. The method of claim 1, wherein the subject is administered once a week (QW) with about 200 mg or twice a week (BIW) with about 100 mg of the compound selected from: 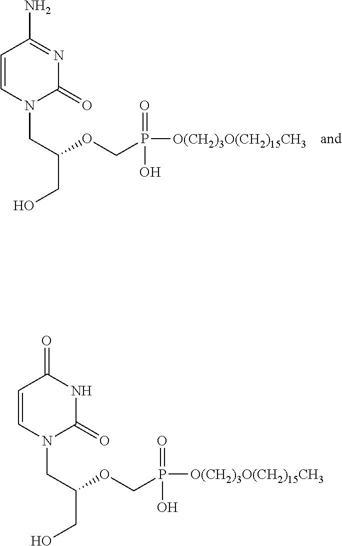
or a pharmaceutically acceptable salt thereof.

10. A method of slowing or reducing the spread of VZV in a subject in need thereof, said method comprising orally administering to said subject a pharmaceutical composition comprising a therapeutically effective dose of a compound selected from: 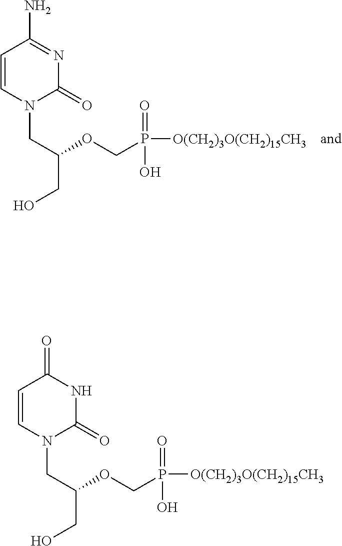
or a pharmaceutically acceptable salt thereof.

11. The method of claim 10, wherein the subject is administered once a week (QW) with about 200 mg or twice a week (BIW) with about 100 mg of the compound or a pharmaceutically acceptable salt thereof.

12. The method of claim 10, wherein the subject is administered 1.25 mg/kg, 2.5 mg/kg, 5.0 mg/kg, 10 mg/kg, or 20 mg/kg of one of the two compounds on day 1, 2, or 4 after post-hematopoietic stem cell transplant (HSCT).

13. The method of claim 4, wherein the subject is administered once a week (QW) with about 200 mg or twice a week (BIW) with about 100 mg of the compound or a pharmaceutically acceptable salt thereof.
